SEQUENCE LISTING (20 sequences)

SEQ ID NO: 1 - DNA, 1149 bp, Vibrio natriegens
  CDS (1)..(1149)
atgactagtgcattttttatccctacggtaaacctcatgggcgcaggctgtcttaaagacgccgcggacagtattcaatcacaaggctttaaaaaagggttgatcgttaccgataaaatccttaaccaaatcggtgtggttgagcaggttcaaggccttcttaacgaaagaagcgtcgcaaccgtagtgttcgatggcactcagccaaacccaacgattaacaacgttaacgacggcttagcactattaaaagaaaacgagtgtgactttgtaatctcacttggcggtggctcaccacacgactgtgcgaaaggtatcgcacttgttgctgcaaacggtggacacatttctgactacgagggtgttgatcaatctgcaaaaccaatgctgccactaatcgccatcaatacgactgcgggtactgcatcggaaatgactcgtttctgtatcatcaccgatgaagaacgtcacattaagatggcaatcgtagataagcacaccacgccattgatctcagtaaacgacgctgagttaatgcttgcaaaaccagcgtctctgaccgcggcaacgggtatggacgcgctgactcacgcgattgaagcgtacgtttctattgcggcaacaccaattacggatgcagttgcgattaaagcgattgagttaattcaagcgcacctgcgcacagcagtgaaaaacggtgaaaatctggaagctcgtgaacaaatggcgtacgcgcagtttatggcgggtatggcgtttaacaacgcttcactgggttacgtccacgcaatggctcaccagttaggtggtttctacgatttaccacacggtgtgtgtaatgccatcctgcttcctcacgtacaacgctacaacgcgcaagtctgccctgaacgtctgactgatgtggcgaaagcaatgggcgtcaacgtggaaggcatgtcagcagagcaaggtgcagcagccgcgatcgacgcgatagttacactagcgaaagatgttggtattcctgctggcatcaaggagcttggcgcgaaactagaagatattccaacgctggctgataacgcacttaaagacgcgtgtggtttcactaaccctaaacaagcgactcacgaagagatctctgcaatattcgaagcggcaatgtaa

SEQ ID NO: 2 - protein, 382 aa, Vibrio natriegens
MTSAFFIPTVNLMGAGCLKDAADSIQSQGFKKGLIVTDKILNQIGVVEQVQGLLNERSVATVVFDGTQPNPTINNVNDGLALLKENECDFVISLGGGSPHDCAKGIALVAANGGHISDYEGVDQSAKPMLPLIAINTTAGTASEMTRFCIITDEERHIKMAIVDKHTTPLISVNDAELMLAKPASLTAATGMDALTHAIEAYVSIAATPITDAVAIKAIELIQAHLRTAVKNGENLEAREQMAYAQFMAGMAFNNASLGYVHAMAHQLGGFYDLPHGVCNAILLPHVQRYNAQVCPERLTDVAKAMGVNVEGMSAEQGAAAAIDAIVTLAKDVGIPAGIKELGAKLEDIPTLADNALKDACGFTNPKQATHEEISAIFEAAM

SEQ ID NO: 3 - DNA, 28 bp, Artificial
  primer
aaagaattcgacgtccacatatacctgc

SEQ ID NO: 4 - DNA, 28 bp, Artificial
  primer
aaagaattcgacgtcaatgccaatagga

SEQ ID NO: 5 - DNA, 29 bp, Artificial
  primer
aaagtcgacgaggaaatggatgagaccag

SEQ ID NO: 6 - DNA, 30 bp, Artificial
  primer
tctgattcgaattggattgctccgtcgttc

SEQ ID NO: 7 - DNA, 29 bp, Artificial
  primer
aaagcatgcttcgagcctttctttatcag

SEQ ID NO: 8 - DNA, 30 bp, Artificial
  primer
gcaatccaattcgaatcagagttcactctc

SEQ ID NO: 9 - DNA, 17 bp, Artificial
  primer
tatcgaccagttgcatg

SEQ ID NO: 10 - DNA, 17 bp, Artificial
  primer
ttcgcttcgccaacatc

SEQ ID NO: 11 - DNA, 1779 bp, Vibrio natriegens
  CDS (526)..(1710)
agtacgcgttatacgactcactatagggagatctagaaggtgctgacagcaaaaacctacgtactatggttgatgatatcaagaaccaaatgggttctggtgttgtacttctggctaacatcacgggtgacaaagttggtttgatcgctggtgtaactaaagatctggttggcaaagtcaaagcgggtgatttagttaaaatggtagcagagcaggttggtggtaaaggcggcggtcgtcctgatatggcgcaagctggcggtactgacgtagcggcacttccagaagcaatcaaaactgttcagccttggctagaagagcgcttgtaacaagctataaataacaaatatacgctcaatcaatttgttttgattgggcgtaatttttttaagttgaccaagtggtttaatggaccagagattaagctggcccactaagctgcttggtttttcttgtaactaccacttatataaacagaatgtatgtggtccgaatgagactttggtcttgggaaggtgaagactggtgaaaaagccccttatcgtgcaaaagtttggcggaacctctgtgggttcaattgaaagaatccaccaagttgctgaacacatcattaaggcgaaaaatgatggtaatcaagttgttgtagttgtgtctgctatgtcaggcgaaaccaataggcttatggacctagctaaacaggtagatagcgttcctacagcccgagaacttgatgttttgctctctgctggtgagcaagtgtcgatggcactgttggcgatgacactgaacaaaatgggtcatcctgcacgctcacttaccggagcacaggcgaacattgtgactgataaccagcacaatgacgcaacgattaagcacattgatactacgagagtgatggcattgcttgaacaagagcatgtcgttatcgtcgcaggttttcagggtgtgaatgaaaatggggatatcaccacattaggtcgaggaggctcagatacgagcgcagtgacactggctggtgcactacgtgctgatgagtgtcaaatctttaccgacgttgatggtatttatacttgtgacccacgtgtagtaaaaacggcgcaaaaaatggcagtgattgactttccttcaatggaagcaatggcaagccgtggtgctaaagttttacatttaccttcggtgcaatatgcatggaagaacaatgtaccactacgcgtgctttctacattcgatgtgaatgaaggtagtttagttaagggagagattggcacacaagccatctctggtatagctattcaacgagaccttgccattattgaagtagataaagaacatttgtccagtgctacaaagcaatgtcagatgctaggtatcgatgtctggaatgtgatcgaggaaacagaacggacaggtatcatgataaaacaagatgcatgtgccaagttcgatctggtgttcagcgataaaatccgtaatagtgaaatggtaagcctgttgaccgcagtagggcttgaggccaatggaatggtcgagcatgcatgcgatttgctggctgagcaagatattgcgatcaatttctgcgcaacgaatgcactcactatgatgctagtattatctcctgactgtgtggatatggcggcaaacattcttcatgatgcttacattacgtctagtgaagcgttaagcattcagcaaaaacatgccctaatagggtgatttcgctaacaaggtagtttacgaaaatataacttttgttggataatagcgtgatagcaagaaaat

SEQ ID NO: 12 - protein, 395 aa, Vibrio natriegens
VKKPLIVQKFGGTSVGSIERIHQVAEHIIKAKNDGNQVVVVVSAMSGETNRLMDLAKQVDSVPTARELDVLLSAGEQVSMALLAMTLNKMGHPARSLTGAQANIVTDNQHNDATIKHIDTTRVMALLEQEHVVIVAGFQGVNENGDITTLGRGGSDTSAVTLAGALRADECQIFTDVDGIYTCDPRVVKTAQKMAVIDFPSMEAMASRGAKVLHLPSVQYAWKNNVPLRVLSTFDVNEGSLVKGEIGTQAISGIAIQRDLAIIEVDKEHLSSATKQCQMLGIDVWNVIEETERTGIMIKQDACAKFDLVFSDKIRNSEMVSLLTAVGLEANGMVEHACDLLAEQDIAINFCATNALTMMLVLSPDCVDMAANILHDAYITSSEALSIQQKHALIG

SEQ ID NO: 13 - DNA, 3547 bp, Vibrio natriegens
  CDS (486)..(2942)
ttaaattgttgtgcaagagtggttgctctgcattttctttgatctttgccgttttagccaagatcgccatgaatgtgggtggaatgtaactttttctatgcaaagctatttcgggaatgaaatactaagataaatttattaaaaaaattgaattttaatttatttctttgttttcagtgatatatagcagaatttatctctgcaagactggaaaataaacaatttttttgttgactctgttacagaaactcggtaaacgtagggttaagcaaacacacaattcgtagaatattaatttatgccagttaacagcctcgttgtaatgaccaccaccattattattaccgacattacaattgtaggggcaggctgctgagcgaaagaaattcacaaaaaaggcctgtatccaacaagatacaggcctttttttatgctttcttaatacaaaatttagacaagtttaagaagatacactggaggaagggatgcgagtattgaagtttggcggttcatcattggctgatgcagatcgctttttaagagcagcagatatcattgcgaataatgctcaacaggaagaagtcgcagtcgtgctttctgcaccgggtaaaacaactaataaactggttgctgtgattgaaggcgctttgcgaaatggtgaagcagaattacaaattaacgagttagaagaatcgtttaaaacgctttttgccgatatccaagccttggttcctaatctggatgggactggctacgacaatcaagttaaaacctcgctctctcaattacgtcaatttgtccatggcatcagcctgttaggcatgtgtccaaacaatgtgaatgcgcgcatcatcagtaaaggtgaacgtgtttctattcaactaatgaaagcggtacttgaagcaaaaggtcaaaaagccagcctaattgatccggttgaatacctttatgctcaaggcgatcatcttgaagcgatggttgatgttgatatttctacacaaaacttccgccaaaagcctcttcctcaaggtcatgtcaacatcatgcctggttttactgccggcaatgagaaaggagaactggttactctaggtcgtaacggttcagactattcggctgcggttttggctgcttgtcttcgcgccgattgttgtgaaatctggactgacgttgatggcgtatacaactgtgatcctcgcttggtagaagatgcacgactattaaaatcactgagctatcaggaagcgatggagttgtcttacttcggtgcgtccgttctgcacccgaaaaccatcgctccaattgctcaattccacattccatgcttaatcaagaacagttttaacccgcaaggtgcaggtacgctgattggccaagatactggtgaagataacctggctatcaagggtattactactctgaatgacctgaccatggtgaacgtttctgggccaggtatgaaaggcatggttggcatggcgagccgtgtatttggtgctatgtcttcagcgggtgtttcaatcgtactgatcactcagtcatcatcagaatacagcatcagcttctgtatcgaagcagaagataaagcaaaagcgcagcagacactggcagaagcgtttgagcttgaactgaaagatggtctgcttgaaccagtagagtttatcgatgatgttgctatcgtgacccttgttggtgacggcatgcgtacatcacgtggtgtggcttcacgtttcttctcatctcttgctgaagtaaacgtcaacattgttgcaatcgcacagggttcttcagagcgcgctatctcagcggtaatcccagaagataagatctctgaagcaatcaaagcctgtcacatgaacctgttcaattctaaacacttccttgatgttttcgtcgtaggcatcggcggtgttggtggcgagctggtggatcagattgaacgccaacaagctaaactggcagaaaaaggcatcatcattcgcgtatgtggtctggcaaacagtaagggtctgcttctggatagtgaagggctgccactggaacactggcgcgaccgcatgtcttcagccactgaagagttcagcctagcgcgcctgattgcacttgtgcaacgtaaccacatcattaaccctgttttggttgattgtacttcgagtgaagccattgctaaccagtacgctgatttcttggcggcaggtttccacgttgttactccaaataagaaagccaatacagcaagcatggcttactaccaccagcttcgcgatgtagcacgcagctctcgtcgtaaattgatgtacgagacaacggttggtgcgggtttgccggttatcgaaaacctgcagaatctcatttcagcgggtgatgagcttgaacgattcaccggcattttgtctggttcactatcttacatcttcggtaagctagacgaaggcatgagcttgagcgaagcgaccaacatcgcgaaagaaaacggcttcaccgaaccggatcctcgtgatgatctatcgggtatggatgtggctcgtaagcttcttattctggcgcgtgaagcgggaatgtcgctagagttggaagacgttgtggttgatcaagcgctgccaccgggctttgatgattcaggcagtgttgatgagtttatggcgagacttcctgaagctgatgcgtactttaaagatcttgtcgcaaaagccgctgaagagggtaaagttttacgttatgttggtgaaatcaatgatggtaagtgcaaagtcagcattgctatcgttgatgaaaatgatccaatgttcaaaattaaagatggtgagaacgctctggcgttttacagccgttactaccaaccaatcccattggtacttcgtggttacggtgcaggtacggaagttaccgcagcaggcgtattttcagatgtgatgcgtactttaggttggaaactaggggtataacagtaatgagttcaagtgatatggatgtagtggtttatgccccagcatcaattggtaatgtaagcgtaggttttgatgttttgggagcggctgtctctcctatcgatggcaccttattgggtgatcgagtcctagtgaaatctggttctgagccatttagcttgaaaaccgcaggtaacttcgtctcgaagttacctacagaaccaaaagaaaacatcgtttacgactgttgggttgtatttgctcgcgagctagatagaaagggcgcagagctgaaaccattggaaatgacactcgagaagaacatgcctatcggttctggtcttggttccagtgcttgttctattgttgctgcattagatgctttgaaccgttttcacgggcaaccacttaatgaaactgagctgttagcgctaatgggcgagatggaaggtaaaatttctggtggtatccattatgacaacgttgcaccttgttacttaggtggcgtgcagttgatgcttgaagagcttggtatcattagccaggaagtgccatgttttgatgactggtactgggtaatggcttatccgggaatcaaagtgtctactgctgaagctcga

SEQ ID NO: 14 - protein, 819 aa, Vibrio natriegens
MRVLKFGGSSLADADRFLRAADIIANNAQQEEVAVVLSAPGKTTNKLVAVIEGALRNGEAELQINELEESFKTLFADIQALVPNLDGTGYDNQVKTSLSQLRQFVHGISLLGMCPNNVNARIISKGERVSIQLMKAVLEAKGQKASLIDPVEYLYAQGDHLEAMVDVDISTQNFRQKPLPQGHVNIMPGFTAGNEKGELVTLGRNGSDYSAAVLAACLRADCCEIWTDVDGVYNCDPRLVEDARLLKSLSYQEAMELSYFGASVLHPKTIAPIAQFHIPCLIKNSFNPQGAGTLIGQDTGEDNLAIKGITTLNDLTMVNVSGPGMKGMVGMASRVFGAMSSAGVSIVLITQSSSEYSISFCIEAEDKAKAQQTLAEAFELELKDGLLEPVEFIDDVAIVTLVGDGMRTSRGVASRFFSSLAEVNVNIVAIAQGSSERAISAVIPEDKISEAIKACHMNLFNSKHFLDVFVVGIGGVGGELVDQIERQQAKLAEKGIIIRVCGLANSKGLLLDSEGLPLEHWRDRMSSATEEFSLARLIALVQRNHIINPVLVDCTSSEAIANQYADFLAAGFHVVTPNKKANTASMAYYHQLRDVARSSRRKLMYETTVGAGLPVIENLQNLISAGDELERFTGILSGSLSYIFGKLDEGMSLSEATNIAKENGFTEPDPRDDLSGMDVARKLLILAREAGMSLELEDVVVDQALPPGFDDSGSVDEFMARLPEADAYFKDLVAKAAEEGKVLRYVGEINDGKCKVSIAIVDENDPMFKIKDGENALAFYSRYYQPIPLVLRGYGAGTEVTAAGVFSDVMRTLGWKLGV

SEQ ID NO: 15 - DNA, 3214 bp, Vibrio natriegens
  CDS (376)..(2781)
ctgcagaagcaatctttggtggcgaaaatttatcatccaagtcttccacagcaccctggccatgagattgctaaaaagcaacagtctggttttggctctatgttgagctttgagtttgcgggctcgtttgagcagcttaaagcctttgtcaaagagcttcagttgttctcacttgcagagtctctgggcggggtagaaagcttgatttgtcacccagcatctatgacgcatcgcgcgatgggtgaagaagccttggctgaagcgggtgtatcacagcaattgctgcgcctttctgtagggctagaagatgcacaagatcttattgccgacctagaacaagcattcactaaatctgcacagcaataggagttaataatgactgtacaacgtcagctacacaagtttggtggtagcagcctggcaaacccagagtgttacttacgtgtagcgggaattctcaaggaatactctgcagaaaatgacttggtggtggtctctgcagcaggtaaaacaaccaaccgcctgattgagttccttgaaggcttagaaaaagatggtcgaattgcgcacgaagctttgcagagcttgagacagtttcaaatcagtctcattgaagagttattagagggagacgcgcaagaacagctgcttgcctccttacaagatgaattcagtacattagcggaactgacctctccgctgaccgaagcgcaaaaggcggcggtattaggccacggagaagtatggtcttctcgattactggccgctttactgacgcaaaagcaactgcctgctgtagcgcaagatgcgcgagcattcctacgcgcagaagcgggcactcaaccagaagttgatcgtgctcgttcatatccgcttatcaaagaggcactggcacaacatagccacaaacgtgtgatcattaccggcttcatggcacaaaacgataacggtgaaactgttctgcttggccgcaacggttcagactactctgcaaccgtaattggtgcgcttgcagaagtgagcacagtgacgatttggagtgatgtggctggtgtttatagcgcagaccctcgtttggtatcagatgcatgtttactacctctcttaaggctggatgaagcgagtgagctggctcgtttagctgcgccagttctgcacagccgaaccttgcagcctgtggctcaaagcaccatggatctgagccttaaatgcagttacctgccagagtcaggttcgacccgcattgagcgagttctggcttctggtcgcggggcgaaaatcatcacctcactggatgacgtgctattggttcagctagcatttgcgcatggacatgactttgagaaagcacagagtgatgttctggaatccctaaaacgcgttcagttggagccactggcatttgaagctcagcctgaacagcagatattacgactggcatacacagcagaaattgctggcggtgcactcaagtatctacaggaatctgatctggatgcggagattaacctcactgatggttactctctgattgctgccgtaggtgctggtgtgacaactaatgccaaccactgttttggttttcagcaaaagctgaaacactctccggtggaatttatcgctgagacggagtctggtttaagtctggtcgcagtattacgtaacactgataccgaagaactggtacagacggttcacagccagttatttcaggcacagaagcgcgttgctgtcgcattatgcgggaaagggaatatcggttcgagctggctgagtttattcgcttcacagaagaatgaacttgaaaaacgtcatggcatgagctttgacttagtcgcggtggttgatagccaaatgtattggtttgatagccaaggcattgatgcatccagtgtttcagcacgcttcaatgacgaaagtatcgcaaacgatggctcttggttatctcgtcttggtgctttgcaggactacgatgaagctgtggtcctcgatgttaccgccagtcaggaattagcgaaatgttacgttgatatcgctcagcaaggtattcacctaatttcagcgaataaagtcgcaggttcagctgatagtcagtattaccatcaggtccaggatgcgtttgccaaaattggacgtcactggctgtataacgctacggtaggtgccgggttaccaatcaaccatacggtgcgcgatcttcgtgagagtggtgatgagattattgcactctctggtattttctccggtacgctttcatggttattccaacagtttgatggctcagtgccattcagcgagttagtggatttagcctggcagcaaggattaacagaacctgaccctcgctctgacttagatggctctgatgtgatgcgtaagctggttattctggcgcgtgagtctggtctggacatcgagccgggaagcgtaaaggtagagtcgttagttcctgaagaactgcgcacgctcagcttagatgagttttttgataacgccgcactgctgagccagactctgcaagaacgtttgtccaaagcgcagaaaaacgatcaggtactgcgttacgttgcgcgtttagagaaagatggcaaagcgaccgtcggcattgaagccttgtcccgtgagcatgcgctggctaacctactgccgtgtgacaatatttttgctattgagagcaagtggtacaaagacaacccgcttgttatccgcggtcctggcgctggacgtgaagttacggcaggggcgattcagtccgatttaaaccgccttgctgggctattctaaacactcaaaaccccggaattcatccggggttttttattcctgtcttggtgccataattaatctcagaaccgttattggcgaactatttttgtggataaatattagccaacaatcaacttgaactatcctcatcattaacgtgaaaaaaattcataatcacaaggttgacattaaatcgtattcaatacattctgtagacatatagacgtctaaacgtcgatttgagaatttgatttcgtggcgggttgccacagggagagtaagatgggttacacgcacgcaggccatatcgatgccttaaaccagaatatcgctgaattgtcagacaatattaatgtttcatttgagttttttccgccaagcaatgaaaagatggaagagacgctatggaactctgtgcaccgtctaaaaacactcaagcctaagtttg

SEQ ID NO: 16 - protein, 802 aa, Vibrio natriegens
MTVQRQLHKFGGSSLANPECYLRVAGILKEYSAENDLVVVSAAGKTTNRLIEFLEGLEKDGRIAHEALQSLRQFQISLIEELLEGDAQEQLLASLQDEFSTLAELTSPLTEAQKAAVLGHGEVWSSRLLAALLTQKQLPAVAQDARAFLRAEAGTQPEVDRARSYPLIKEALAQHSHKRVIITGFMAQNDNGETVLLGRNGSDYSATVIGALAEVSTVTIWSDVAGVYSADPRLVSDACLLPLLRLDEASELARLAAPVLHSRTLQPVAQSTMDLSLKCSYLPESGSTRIERVLASGRGAKIITSLDDVLLVQLAFAHGHDFEKAQSDVLESLKRVQLEPLAFEAQPEQQILRLAYTAEIAGGALKYLQESDLDAEINLTDGYSLIAAVGAGVTTNANHCFGFQQKLKHSPVEFIAETESGLSLVAVLRNTDTEELVQTVHSQLFQAQKRVAVALCGKGNIGSSWLSLFASQKNELEKRHGMSFDLVAVVDSQMYWFDSQGIDASSVSARFNDESIANDGSWLSRLGALQDYDEAVVLDVTASQELAKCYVDIAQQGIHLISANKVAGSADSQYYHQVQDAFAKIGRHWLYNATVGAGLPINHTVRDLRESGDEIIALSGIFSGTLSWLFQQFDGSVPFSELVDLAWQQGLTEPDPRSDLDGSDVMRKLVILARESGLDIEPGSVKVESLVPEELRTLSLDEFFDNAALLSQTLQERLSKAQKNDQVLRYVARLEKDGKATVGIEALSREHALANLLPCDNIFAIESKWYKDNPLVIRGPGAGREVTAGAIQSDLNRLAGLF

SEQ ID NO: 17 - DNA, 2648 bp, Vibrio natriegens
  misc_feature (862): n = a, t, g or c
ataactacggccagtgccaagcttgcatgcctgcaggtcgactctagaggatccccttgtgttgcttggctaacttacccaaagcttgagcgtcgctgaccgcacctgtcgatgtttccgcgtgaacaaatgctaaaatttttgtgtcaggatgctgctgcagtgcttgttcgactttatcgacagagactggcgcgccccactcgtcatcaaccaagacaacttcaccaccagcacgaacaacgttttcacgcatgcgctcaccaaaaacaccatttcggcatacgataactttgtcacctttctcgataaggttaacaaaacatgcctccatacccgcactacctggagcagaaacggcaatagtaaagtcgttttctgtctggaacgcgtactttagcaattgtttcagctcatccatcattgcgataaacagagggtccaagtgaccaaccgttgggcggctaagcgcttgcagcacttgcggatagatatctgaaggacctggtcccattagggtacggtgagggggaataaagctttggatcggcatgtgagctccttgtttgacgttacttaacccgagcgcggacaatagtttttagaattttgaataagtctcgggcatcaaagaactcacactaatggattttatagtgctcggcaattagccgtaggtattgagttcaaaccactgtcacaatttcactaataaaaaatcttggaatagttgaaatattcattgactttgtgacgccaaaaccgttcaatgtttgagctttatgtggaagaggagcactacccaggtagataatttgtggaaccgcaattccaatacgaattattcagggggagtagtgcncgaggtaagtcaaatttgcagggtttgacttgtcggttgacgtgggttgagtcccatcaactgtcatcagcattgtctgatgaagagcttctgagggtacatatttcaaatatcactctaaatacctctctttttgctctgttcctttcctcactaaatatcggatatattagtttaagaagtcgtgggagaactcagtgagcgcatttaacgtagcaaaattcggtggaaccagtgttgccaatttcgaggcaatgagccgttgtgccgccattattgaaaacaatccaaatactcgccttgtcgttagcagcgcttgctctggtgtaacgaacctattggtagaactggccaatggtgttcaggatcaggaacagcgtgccgagttgctacgtaagctagcagaaattcatgatgacatccttagtcaactaagagatgcagcagaagcaagtgcagaggtttacgcaatccttgatacggtaaccagccttgcagaagcagcttctatccaagctagctctaaactgacagaccatttagttgcgtgtggtgagttaatgtcgacgcacattttggctcaactgatgagagagcgtggtatcaatgcggtgcgttttgatattcgtgatgtgctaagaaccgacgataacttcggacgtgctgagccaaatgtcgaagcgatttctcagctagctcaagagaagttagtccctctatgtcaggagtcagtagtcattactcaaggcttcatcggctcagacgaagaaggcaatacaaccacattaggtcgtggtggcagtgactacagtgccgcactcattgctgaaggcgttaaagcttctggcttagaaatctggactgatgttccgggcatctacacaacagacccacgtattgcgccaaaagcgtctcctattccagagatcagctttagcgaagcgtcagaaatggcaaacttcggtgctaagatcctccacccttctacactcgttcctgctttgcgccacgacattccagtgtttgttggctcttctaaagatccagaagcaggtggcacatggattcgtcaccaagtagaaagctcgccgctataccgagctcttgcattgcgttgcaaccaaaccatggtcactctacgtagtgcaagcatgttccatgcatacggcttcctggctaaagtgttcgagattctggctaaacacaaaatttcagtggacctgattaccacttcagaaatcagtgtttcgttaactctagaccaaacagacacctctggtggtgcaccacaactacctcaagccgtaagagaagagctagaagaattgtgtaaagtggaagtagagcacgacctatgcttggtggctcttatcggaaacaacatgagcggtagcaaaggatacgccaaacaagtattcggtacattagaagactttaacctgcgtatgatttgctacggtgcgagcccgcataacttgtgcttcctactgcacaagtcagaatcgcaacaggcgattcaaaaactgcatgctgagctatttgaaaaataaacgctgttcatctcacaatataaaaagggctgcctgatggcagccctttctctttccgttggattagccgttaatattcggacctaaccatttttctgcttcaagcctgtcccaacctttacgttcagcatagctttctaactgatcttcctggatctgcgcgatcgcaaagtagcgagaatccggatgagagaagtaccaaccagacactgaggcaccagggtacatggcataga

SEQ ID NO: 18 - protein, 450 aa, Vibrio natriegens
VSAFNVAKFGGTSVANFEAMSRCAAIIENNPNTRLVVSSACSGVTNLLVELANGVQDQEQRAELLRKLAEIHDDILSQLRDAAEASAEVYAILDTVTSLAEAASIQASSKLTDHLVACGELMSTHILAQLMRERGINAVRFDIRDVLRTDDNFGRAEPNVEAISQLAQEKLVPLCQESVVITQGFIGSDEEGNTTTLGRGGSDYSAALIAEGVKASGLEIWTDVPGIYTTDPRIAPKASPIPEISFSEASEMANFGAKILHPSTLVPALRHDIPVFVGSSKDPEAGGTWIRHQVESSPLYRALALRCNQTMVTLRSASMFHAYGFLAKVFEILAKHKISVDLITTSEISVSLTLDQTDTSGGAPQLPQAVREELEELCKVEVEHDLCLVALIGNNMSGSKGYAKQVFGTLEDFNLRMICYGASPHNLCFLLHKSESQQAIQKLHAELFEK

SEQ ID NO: 19 - DNA, 2331 bp, Vibrio natriegens
  CDS (344)..(1765)
acggaacacgcacatccactacaaaaaccacttagagtcagttttctgtatgagtggtgaaggcgagattgaagtggtaggtggtgaaacctatccaatcaaaccaggtacactgtacattctggataaaaacgacgagcactacctaagagcatataaaaacaaagaaatggtaatggcgtgcgtattcaacccacctatcacaggtgcggaagtacacgatgagaacggcgtgtaccctcttgttgactaaaactaaatagtcactgtgaggtgggcttccacctcacatttcaattctctcttctttattttttaagttaccaatcaaaaaaggcttaacatgacttttaccgtagaaaaaatcggcggtacttcaatgacagcatttgatgctgttctagacaatattattcttcgtcccaagacaccatacaaccgagtatttgttgtatcggcgtacggtggtatgactgacgcgctattagaatgtaaaaaaaccagtaaagcgggcgtataccaactggttgctaagcgtgatgactcgtgggaagaagcgttggcatacgtagagaatcgtatgttgctgacaaacgagaatattttcgccgatccaatgaatcgaatgcgagcggataagtttattcgttcacgcatttcagaagcgaaaaactgtatcgctaatattttagaaacgtgccagtacggccagttttcactgcgtcactacttgcctcaaatccgtgagtttctttcttcaattggtgaagcgcacagtgcttataacaccgcgttgaaactgaaaaacatgggcattaacgctaagtttgtcgacttgtcaggttgggatacaaccgagccgaagagtcttgatgaatcaatcagtgaagcttttgctgatatcgatgtatcaaaagaactacctatcgtgacgggttatgcatattgtaaagaaggtcttatgcatacgtatgaccgaggctacagtgagatgactttcagccgagttgcttcgatcacgaaagcgaatttagcgataatccacaaggaatatcacttaagttctgctgacccacgtgttgttggtcctgagaaagtattgccaattggcagcacgaactacgatgtggctgaccagctggctaacctgggaatggaagctattcaccctaacgcagcagcgggtttgcgcgaaagtggtatcgaactgcagattaaaaacacatttgaacctgaacatgaaggtactttgatttcttctggttaccgtccagaggaagacaaagtggagatcatcgcgggtaagcagaaagtgtttgcgttgcatctttttgaccaagcgatggttggtaaagtagataacgtgagttacgagctgatggaaatcatctcagatgcacacgtgacattggttggtaaagaaatgaacgccaactcgattacttactacctgggcggtaatgcagacagcttaaacaaagttctgtataaagcggaaaaatgttacccgaaagcatcaattaaaggccgtatggtagcgttgatctcagcgattggttctcagattgacaccaacaaaaccttggcgaaaggtgtattggcactgatgaatagtggtgtgacgccagtggctctgcactcatcattacgaaatgttaatgtacaattcgttgtgggtgataaagagtatcagcgagcaatttgtgcactgcacgatgagttcttcgaaccggttgagaatgctgagtcgatagaagacgttgcgtaagtcaattacacaacaagacgtgatacaaagggagcctttctaggctcctttgttgtttgtggctttactgtgtttatttcggcaatgaaaagcggcttatttggacgataacacacgggctgtgtgcgtatgcttatccgttctttagcttacactttctgtcacgacaggatttgagtggcgtctggctatccaatacgcgataccgccccagaacacggttagcagccaaagggcagtccagtgtttagccacttgttgccatgttgcgcccatttgatttaaggcgagaaagcccttaatcgcccaagtactcgggcttaaatcagcaacccaaagcaggggagccggaatggattctactggccaaatgaaaccggcaaggaagataagtggcattgagctgaccaatacgactaaggtgaccagttctctgcgtggtaaaaggtaaccgagccaaaaacccaaaccacagcagctcaacagaaacggaaccagcaaggaaagcagttcagttgcctgagcgatgtgattacgctacctttcaaagctggcaccaaaat

SEQ ID NO: 20 - protein, 474 aa, Vibrio natriegens
MTFTVEKIGGTSMTAFDAVLDNIILRPKTPYNRVFVVSAYGGMTDALLECKKTSKAGVYQLVAKRDDSWEEALAYVENRMLLTNENIFADPMNRMRADKFIRSRISEAKNCIANILETCQYGQFSLRHYLPQIREFLSSIGEAHSAYNTALKLKNMGINAKFVDLSGWDTTEPKSLDESISEAFADIDVSKELPIVTGYAYCKEGLMHTYDRGYSEMTFSRVASITKANLAIIHKEYHLSSADPRVVGPEKVLPIGSTNYDVADQLANLGMEAIHPNAAAGLRESGIELQIKNTFEPEHEGTLISSGYRPEEDKVEIIAGKQKVFALHLFDQAMVGKVDNVSYELMEIISDAHVTLVGKEMNANSITYYLGGNADSLNKVLYKAEKCYPKASIKGRMVALISAIGSQIDTNKTLAKGVLALMNSGVTPVALHSSLRNVNVQFVVGDKEYQRAICALHDEFFEPVENAESIEDVA

(19) United States
(12) Patent Application Publication
(10) Pub. No.: US 2012/0156736 A1
(43) Pub. Date: Jun. 21, 2012

(54) METHOD FOR PRODUCING L-LYSINE USING A VIBRIO BACTERIUM

(76) Inventors: Ippei Inoue, Kawasaki-shi (JP); Hisashi Yasueda, Kawasaki-shi (JP)

(21) Appl. No.: 13/364,637

(22) Filed: Feb. 2, 2012

Related U.S. Application Data
(63) Continuation of application No. PCT/JP2010/060972, filed on Jun. 28, 2010.

(30) Foreign Application Priority Data
Aug. 3, 2009 (JP) 2009-180819

(51) Int. Cl. C12P 13/08; C12N 1/20

(52) U.S. Cl. 435/115; 435/252.1

(57) ABSTRACT

L-Lysine is produced by culturing in a medium a Vibrio bacterium which has an ability to produce L-lysine, and has been modified so that an activity of a protein encoded by the fucO gene is reduced to produce and accumulate L-lysine in the medium or cells of the bacterium, and collecting L-lysine from the medium or cells.

This application is a Continuation of, and claims priority under 35 U.S.C. § 120 to, International Application No. PCT/JP2010/060972, filed Jun. 28, 2010, and claims priority therethrough under 35 U.S.C. § 119 to Japanese Patent Application No. 2009-180819, filed Aug. 3, 2009, the entireties of which are incorporated by reference herein. Also, the Sequence Listing filed electronically herewith is hereby incorporated by reference (File name: 2012-02-02T_US-472_Seq_List; File size: 82 KB; Date recorded: Feb. 2, 2012).

### BACKGROUND OF THE INVENTION

1. Field of the Invention

The present invention relates to a method for producing L-lysine using a Vibrio bacterium. L-Lysine, an industrially useful L-amino acid, is used as an additive for animal feed, ingredient in health foods, amino acid infusions, and so forth.

2. Brief Description of the Related Art

L-lysine is typically produced by fermentation using a coryneform bacterium belonging to the genus Brevibacterium, Corynebacterium, or the like, a bacterium belonging to the genus Bacillus, Escherichia, Streptomyces, Methylobacillus, or the like, or a filamentous fungus belonging to the genus Penicillium, or the like.

Many strains are used in methods for producing L-lysine by fermentation using a microorganism, including using an auxotroph derived from a wild-type strain, a metabolic regulation mutant strain derived from a wild-type strain as a strain resistant to any of various drugs, a strain having properties of both auxotroph and metabolic regulation mutants, and so forth. Furthermore, microorganisms that can efficiently produce L-lysine have been bred using recombinant DNA techniques (European Patent Laid-open No. 0857784, Japanese Patent Laid-open (KOKAI) No. 11-192088, International Patent Publications WO00/53726 and WO96/17930).

Although productivity of L-lysine has been considerably increased by such breeding of microorganisms and improvement of the production methods as described above, development of even more inexpensive and efficient L-lysine production methods is desirable in order to respond to further increase of the demand in future.

L-Amino acid production methods based on direct fermentation have a problem that the action of a microorganism is reduced by increase of osmotic pressure due to accumulation of L-amino acid in the medium, and thus the productivity cannot be maintained for a long period of time. As a method for solving this problem, production of an L-amino acid such as L-lysine by direct fermentation using a Vibrio bacterium has been disclosed (International Patent Publication WO2008/093829).

As a method for producing L-lysine in which decomposition of L-lysine accumulated in the medium can be reduced, methods using a microorganism of which L-lysine decomposition ability is reduced, in particular, a microorganism belonging to the genus Escherichia in which the cadA gene or the ldcC gene is deleted, or expression amount or enzymatic activity of the product thereof is decreased have been reported (S. Meng, and G. N. Bennett, J. Bacteriol., 174, 2659-2669 (1992), International Patent Publication WO96/17930).

It is known that a cadA homologue gene is present in Vibrio bacteria such as Vibrio cholerae, Vibrio parahaemolyticus and Vibrio vuinificus (D. S. Merrell, and A. Camilli, Mol. Microbiol., 34, 836-849 (1999), Y. Tanaka, et al., J. Appl. Microbiol., 104, 1283-1293 (2007), J. E. Rhee, et al., FEMS Microbiol. Lett., 208, 245-251 (2002)). However, it has not been reported whether the ability to decompose L-lysine is attenuated, reduced, or eliminated by disruption of such a cadA homologue gene in these Vibrio bacteria. Moreover, presence of an L-lysine decomposition pathway other than the L-lysine decomposition pathway involving a cadA homologue gene product has not been reported in Vibrio bacteria.

### SUMMARY OF THE INVENTION

An aspect of the present invention is to provide a method for efficiently producing L-lysine using a Vibrio bacterium. In particular, the inventors of the present invention have already found a phenomenon that production of L-lysine decreases as the culture progresses in the case of Vibrio bacteria, and one aspect of the present invention is to reduce this decrease of L-lysine.

A gene coding for a factor involved in L-lysine decomposition in Vibrio bacteria has been found, and it has been found that, by disrupting that gene, decomposition of the L-lysine that is produced can be suppressed, and L-lysine can be more efficiently produced.

It is an aspect of the present invention to provide a method for producing L-lysine, which comprises culturing a Vibrio bacterium having an ability to produce L-lysine in a medium to produce and accumulate L-lysine in the medium or cells of the bacterium, and collecting L-lysine from the medium or cells, wherein the Vibrio bacterium has been modified so that an activity of a protein encoded by the fucO gene is reduced.

It is a further aspect of the present invention to provide the method as described above, wherein the activity of the protein is reduced by introducing a mutation into a coding region of the fucO gene and/or an expression control region of the gene.

It is a further aspect of the present invention to provide the method as described above, wherein the fucO gene on the bacterium's chromosome is disrupted.

It is a further aspect of the present invention to provide the method as described above, wherein the protein is selected from the group consisting of:
(A) a protein comprising the amino acid sequence shown in SEQ ID NO: 2, and
(B) a protein comprising the amino acid sequence shown in SEQ ID NO: 2, but wherein one or several amino acid residues are substituted, deleted, inserted or added,

wherein the reduction of the activity in the bacterium improves the ability to produce L-lysine.

It is a further aspect of the present invention to provide the method as described above, wherein the fucO gene is a DNA selected from a group consisting of:
(a) a DNA comprising the nucleotide sequence of SEQ ID NO: 1, and
(b) a DNA which hybridizes with the nucleotide sequence of SEQ ID NO: 1 or a probe that can be prepared from the nucleotide sequence under stringent conditions, and codes for a protein, wherein the reduction of the activity in the bacterium improves the ability to produce L-lysine.

It is a further aspect of the present invention to provide the method as described above, wherein an activity is enhanced of an enzyme selected from the group consisting of dihydrodipicolinate synthase, aspartokinase, dihydrodipicolinate reductase, diaminopimelate dehydrogenase, and combinations thereof.

It is a further aspect of the present invention to provide the method as mentioned above, wherein the Vibrio bacterium is Vibrio natriegens.

It is a further aspect of the present invention to provide the method as mentioned above, wherein the medium comprises glycerol as a carbon source.

It is a further aspect of the present invention to providea Vibrio bacterium which has an ability to produce L-lysine, and has been modified so that an activity of a protein encoded by the fucO gene is reduced.

It is a further aspect of the present invention to provide the Vibrio bacterium as mentioned above, which is Vibrio natriegens.

### BRIEF DESCRIPTION OF THE DRAWINGS

FIG. 1 shows suppression of L-lysine decomposition when the fucO gene is deficient: (a) change of OD660 of culture medium diluted 51-fold over the period of time, (b) change of glucose concentration in the medium over the period of time, and (c) change of L-lysine concentration in the medium over the period of time. Each graph includes curves plotted with averages of results for three samples, and indicates standard deviations.

FIG. 2 shows L-lysine production from glucose by V. natriegens 28-15 ΔfucO/pCABD2: (a) OD660 of culture medium diluted 51-fold, (b) glucose concentration in the medium, (c) L-lysine concentration in the medium, and (d) L-lysine yield based on consumed saccharide 40 hours after the start of the culture. In the graphs, the indications of 0 to 8% NaCl mean that the graphs show averages and standard deviations of the results obtained by culture in the MS culture media containing 0 to 8% (w/v) NaCl.

FIG. 3 shows L-lysine production from glycerol by V. natriegens 28-15 ΔfucO/pCABD2: (a) change of OD660 of culture medium diluted 51-fold over the period of time, (b) change of glycerol concentration in the medium over the period of time, (c) change of L-lysine concentration in the medium over the period of time, and (d) L-lysine yield based on consumed glycerol 40 hours after the start of the culture. In the graphs, the indications of 0% NaCl, 2% NaCl and 4% NaCl mean that the graphs show averages and standard deviations of the results obtained by culture in the MS culture media containing glycerol as a carbon source and 0 to 4% (w/v) NaCl.

FIG. 4 shows L-lysine production from glycerol by V. natriegens 28-15 ΔfucO/pCABD2 and V. natriegens VLD01/pCABD2: (a) change of OD660 of culture medium diluted 50-fold, (b) change of glycerol concentration in the medium over the period of time, (c) change of L-lysine concentration in the medium over the period of time, (d) L-lysine yield based on consumed glycerol 24 hours after the start of the culture.

### DETAILED DESCRIPTION OF EXEMPLARY EMBODIMENTS

<1> Vibrio bacterium

The bacterium of the presently disclosed subject matter is a Vibrio bacterium which has an ability to produce L-lysine and has been modified so that an activity of a protein encoded by the fucO gene is reduced.

The ability to produce L-lysine can mean an ability of the bacterium to produce and accumulate L-lysine in a medium or cells thereof in such a degree that L-lysine can be collected from the medium or cells, when the bacterium is cultured in the medium. That is, the bacterium is a Vibrio bacterium that can produce L-lysine by fermentation using a saccharide or the like as a carbon source (also called direct fermentation). Specifically, when a Vibrio bacterium which has been modified to have reduced activity of the protein encoded by the fucO gene is cultured under appropriate conditions, for example, if 1.5 times or more, twice or more, or 3 times or more, of L-lysine is accumulated, the bacterium has the ability to produce L-lysine.

“L-lysine” includes L-lysine in free form or a salt thereof such as sulfate, hydrochloride and carbonate.

The Vibrio bacterium having the ability to produce L-lysine may be a bacterium inherently having the ability to produce L-lysine, or may be a bacterium obtained by modifying a Vibrio bacterium such as those described later using mutagenisis methods or DNA recombination techniques so that the bacterium acquires the ability to produce L-lysine. The Vibrio bacterium of the presently disclosed subject matter may have an ability to produce another amino acid, for example, L-threonine, L-glutamic acid, or the like, in addition to the ability to produce L-lysine.

Hereinafter, the Vibrio bacteria will be further described.

The Vibrio bacteria are facultative anaerobic gram-negative bacteria belonging to the family Vibrionaceae of γ-Proteobacteria, and are motile bacteria with a single polar flagellum seen in fresh water or seawater. The Vibrio bacterium can be a nonpathogenic Vibrio bacterium, and Vibrio bacteria for which pathogenicity is not known are listed in Biosafety level 1 (Biosafety in Microbiological and Biomedical Laboratories (BMBL), 4th Edition, published by Office of Health and Safety (OHS)), and such Vibrio bacteria as mentioned below can be used.

Vibrio abalonicus ATCC 27390

Vibrio adaptatus ATCC 19263

Vibrio aerogenes ATCC 700797

Vibrio aestuarianus ATCC 35048

Vibrio alginolyticus ATCC 14582

Vibrio algosus ATCC 14390

Vibrio anguillarum ATCC 43305

Vibrio calviensis ATCC BAA-606

Vibrio campbellii ATCC 25920

Vibrio carchariae ATCC 35084

Vibrio coralliilyticus ATCC BAA-450

Vibrio costicola ATCC 43147

Vibrio cyclitrophicus ATCC 700982

Vibrio cyclosites ATCC 14635

Vibrio diazotrophicus ATCC 33466

Vibrio fischeri ATCC 25918

Vibrio gazogenes ATCC 29988

Vibrio halioticoli ATCC 700680

Vibrio harveyi ATCC 14126

Vibrio hispanica ATCC 51589

Vibrio ichthyoenteri ATCC 700023

Vibrio iliopiscarius ATCC 51760

Vibrio lentus ATCC BAA-539

Vibrio liquefaciens ATCC 17058

Vibrio logei ATCC 15382

Vibrio marinagilis ATCC 14398

Vibrio marinofulvus ATCC 14395

Vibrio marinovulgaris ATCC 14394

Vibrio mediterranei ATCC 43341

Vibrio metschnikovii ATCC 7708

Vibrio mytili ATCC 51288

Vibrio natriegens ATCC 14048

Vibrio navarrensis ATCC 51183

Vibrio nereis ATCC 25917

Vibrio nigripulchritudo ATCC 27043

Vibrio ordalii ATCC 33509

Vibrio orientalis ATCC 33933

Vibrio pectenicida ATCC 700783

Vibrio pelagius ATCC 33504

Vibrio penaeicida ATCC 51841

Vibrio ponticus ATCC 14391

Vibrio proteolyticus ATCC 53559

Vibrio psychroerythrus ATCC 27364

Vibrio salmonicida ATCC 43839

Vibrio shiloii ATCC BAA-91

Vibrio splendidus ATCC 33125

Vibrio tyrosinaticus ATCC 19378

Vibrio viscosus ATCC BAA-105

Vibrio wodanis ATCC BAA-104

Beneckea pelagia ATCC 25916

Listonella anguillarum ATCC 19264

Beneckea pelagia and Listonella anguillarum are closely related to Vibrio bacteria, and some strains thereof are classified as Vibrio bacteria according to the current classification (Thompson F. L. et al., Microbiol. Mol. Biol. Rev., 23, 403-431 (2004) and Macian M. C. et al., Syst. Appl. Microbiol., 23, 373-375 (2000)). Therefore, such bacteria can also be used as the Vibrio bacterium.

Among these, Vibrio natriegens is one example. Vibrio natriegens is an oceanic facultative anaerobic bacterium belonging to the family Vibrionaceae of γ-Proteobacteria, and it was isolated in 1958 as an uronic acid-oxidizing bacterium (Payne, W. J., J. Bacteriology, 76, 301 (1958)). At first, the bacterium was considered to belong to the genus Pseudomonas of γ-Proteobacteria, but the bacterium was re-classified into the genus Beneckea and then incorporated into the genus Vibrio along with other bacteria belonging to the genus Beneckea. That bacterium is classified as Biosafety level 1 in ATCC, and classified as Risk Group 1 (German classification) in the German National Resource Center for Biological Material (DSMZ), and pathogenicity is not known for the bacterium.

As Vibrio natriegens, the Vibrio natriegens ATCC 14048 strain (NBRC 15636 strain) can be used.

The Vibrio bacteria described above are available from, for example, American Type Culture Collection (Address: P.O. Box 1549, Manassas, Va. 20108, United States of America). That is, accession numbers are given to the strains, respectively, and the strains can be ordered using these accession numbers (refer to www.atcc.org/). The accession numbers of the strains are listed in the catalogue of the American Type Culture Collection. The Vibrio natriegens ATCC 14048 strain is also stored at the National Institute of Technology and Evaluation, Biological Resource Center (NITE NBRC, 2-5-8 Kazusakamatari, Kisarazu-shi, Chiba-ken, 292-0818, Japan), with a number of NBRC 15636, and can also be provided from that institute.

The Vibrio bacteria can grow well under high osmotic pressure during a period when the product accumulates at a high level at a later stage of amino acid fermentation, or under high osmotic pressure induced by a high sugar concentration, in contrast to microorganisms that have been conventionally used for production of an L-amino acid (such as Escherichia coli and coryneform bacteria), which cannot sufficiently grow under such conditions. The “high osmotic pressure” can be, for example, not lower than 925 mOsm, not lower than 1100 mOsm, or not lower than 1500 mOsm. The upper limit of osmotic pressure is not particularly limited so long as the amino acid fermentation is possible, and is, for example, 2000 mOsm. In addition, the expression “the bacteria can grow well under high osmotic pressure” can mean that the growth rate is maintained to be, for example, not lower than 50% of the maximum growth rate at 1100 mOsm, at which the growth rate of an E. coli wild-type strain, for example MG1655 strain (ATCC 47076), decreases to 50% or less of the maximum growth rate. In particular example, the growth rate is maintained to be not lower than about 90% of the maximum growth rate.

<2> Method for Imparting the Ability to Produce L-lysine to Vibrio bacterium

A Vibrio bacterium having an ability to produce L-lysine can be obtained by imparting an ability to produce L-lysine to a wild-type strain of any Vibrio bacteria as described above. To impart an ability to produce L-lysine, methods conventionally employed in the breeding of coryneform bacteria, Escherichia bacteria etc. (see “Amino Acid Fermentation”, Gakkai Shuppan Center Ltd.) can be used. Such methods include acquiring auxotrophic mutant strains, strains resistant to an analogue of L-amino acid such as L-lysine, or a metabolic regulation mutant strains, constructing a recombinant strain in which activity of an L-lysine biosynthesis enzyme is increased, and so forth. Activity of an L-lysine biosynthesis enzyme can be enhanced by increasing the copy number of a gene coding for the enzyme or modifying an expression regulatory sequence such as promoter of the gene. In the breeding of L-lysine-producing bacteria, the impartation of such properties as auxotrophy, analogue resistance and metabolic regulation mutation may be combined with the enhancement of an L-lysine biosynthesis enzyme. Methods for imparting an ability to produce L-lysine will be exemplified below.

L-Lysine-producing bacteria can be bred as, for example, mutant strains that are auxotrophic for L-homoserine, or L-threonine and L-methionine (Japanese Patent Publication (KOKOKU) Nos. 48-28078 and 56-6499), mutant strains that are auxotrophic for inositol or acetic acid (Japanese Patent Laid-open Nos. 55-9784 and 56-8692), or mutant strains that are resistant to oxalysine, lysine hydroxamate, S-(2-aminoethyl)-L-cysteine, γ-methyllysine, α-chlorocaprolactam, DL-α-amino-ε-caprolactam, α-amino-lauryllactam, aspartate analogues, sulfa drugs, quinoids, or N-lauroylleucine. In a particular example, a strain is bred to be resistant to the L-lysine analogue S-(2-aminoethyl)-L-cysteine (AEC).

Examples of the mutagenesis method for obtaining a mutant strain of a Vibrio bacterium include ultraviolet irradiation, and treatment with a mutagen used for conventional mutagenesis such as N-methyl-N′-nitro-N-nitrosoguanidine (NTG) and nitrous acid. A Vibrio bacterium having an ability to produce an L-amino acid can also be obtained by selecting a naturally occurring mutant of a Vibrio bacterium.

An L-amino acid analogue resistant mutant strain can be obtained by, for example, inoculating Vibrio bacteria subjected to a mutation treatment on agar media containing the L-amino acid analogue at various concentrations and selecting a strain that forms a colony.

An auxotrophic mutant strain can be obtained by growing Vibrio bacteria on an agar medium containing a specific nutrient such as an L-amino acid so colonies form, then replicating the colonies on an agar medium without the nutrient, and selecting the strain that does not grow on the medium not containing the nutrient.

Examples of L-lysine-producing strain of Vibrio natriegens resistant to AEC include the Vibrio natriegens 28-15 strain (PERM BP-10946). This strain, designated as AJ110593, was deposited at the National Institute of Advanced Industrial Science and Technology, International Patent Organism Depositary (Tsukuba Central 6, 1-1, Higashi 1-Chome, Tsukuba-shi, Ibaraki-ken, 305-8566, Japan) on Oct. 24, 2006, and assigned an accession number of FERM P-21066. It was then converted to an international deposit, and assigned an accession number of FERM BP-10946.

Methods for imparting or enhancing an ability to produce L-lysine by enhancing the activity of an L-lysine biosynthesis enzyme will be exemplified below.

For example, an ability to produce L-lysine can be imparted by enhancing the dihydrodipicolinate synthase activity and/or the aspartokinase activity.

Enhancement of the dihydrodipicolinate synthase activity and/or the aspartokinase activity of a Vibrio bacterium can be attained by constructing a recombinant DNA by ligating a gene fragment coding for dihydrodipicolinate synthase and/or a gene fragment coding for aspartokinase to a vector that functions in Vibrio bacteria, such as a multi-copy type vector, and introducing the DNA into a host Vibrio bacterium for transformation. The activities of these enzymes are enhanced as a result of an increase in the copy numbers of the genes coding for dihydrodipicolinate synthase and/or aspartokinase in the cells of the transformant strain. Hereinafter, dihydrodipicolinate synthase may be abbreviated as DDPS, aspartokinase may be abbreviated as AK, and aspartokinase III may be abbreviated as AKIII

Any microorganism may be used to obtain the genes coding for DDPS and AK so long as the chosen microorganism can express the DDPS and AK activities in a microorganism belonging to the genus Vibrio. The microorganism can be a wild-type strain or a mutant strain that is derived from a wild-type strain. Specific examples thereof include the E. coli (Escherichia coli) K-12 strain and the Vibrio natriegens ATCC 14048 strain (NBRC 15636). The nucleotide sequences of both genes coding for DDPS (dapA, Richaud, F. et al. J. Bacteriol., 297 (1986)) and AKIII (lysC, Cassan, M., Parsot, C., Cohen, G. N. and Patte, J. C., J. Biol. Chem., 261, 1052 (1986)) derived from an Escherichia bacterium have been already elucidated. Therefore, these genes can be obtained by synthesizing primers on the basis of the nucleotide sequences of the genes and performing PCR using a chromosomal DNA of a microorganism such as the E. coli K-12 strain or the Vibrio natriegens ATCC 14048 strain as a template.

Genes of Vibrio bacteria can also be obtained by using the following GenBank database information.

Vibrio cholerae O1 biovar eltor str. N16961 chromosome I, complete sequence: AE003852

Vibrio cholerae O1 biovar eltor str. N16961 chromosome II, complete sequence: AE003853

Vibrio parahaemolyticus RIMD 2210633 chromosome I, complete sequence: BA000031

Vibrio parahaemolyticus RIMD 2210633 chromosome II, complete sequence: BA000032

Vibrio fischeri ES 114 chromosome I, complete sequence: CP000020

Vibrio fischeri ES 114 chromosome II, complete sequence: CP000021

Vibrio vulnificus CMCP6 chromosome I, complete sequence: AE016795

Vibrio vulnificus CMCP6 chromosome II, complete sequence: AE016796

Vibrio vulnificus YJO16 chromosome I, complete sequence: BA000037

Vibrio vulnificus YJO16 chromosome II, complete sequence; BA000038

DDPS and AK can be not subject to feedback inhibition by L-lysine. It is known that wild-type DDPS derived from a Vibrio bacterium is not subject to feedback inhibition by L-lysine, and wild-type AKIII derived from a Vibrio bacterium is subject to suppression and feedback inhibition by L-lysine. Therefore, dapA and lysC to be introduced into a Vibrio bacterium can code for DDPS and AK having a mutation for desensitizing them to the feedback inhibition by L-lysine.

However, DDPS and AK may not necessarily be mutants. For example, it is known that DDPS derived from a Corynebacterium bacterium is not originally subject to the feedback inhibition by L-lysine.

Homologues of a gene coding for aspartokinase may be present, and the source of the gene is not limited so long as the gene codes for a protein having the aspartokinase activity.

For example, examples of the AK gene of Vibrio natriegens include the AKO gene, thrA gene, metL gene, lysC gene, and putative-AK gene.

SEQ ID NO: 11 is the nucleotide sequence of the region including the AKO gene of Vibrio natriegens. It is estimated that, in SEQ ID NO: 11, GTG (nucleotide numbers 526 to 528), GTG (nucleotide numbers 544 to 546), or GTG (nucleotide numbers of 568 to 570) can be the initiation codon, and TGA (nucleotide numbers 1711 to 1713) can be the stop codon. Therefore, a DNA having the nucleotide sequence of the nucleotide numbers 526 to 1710 in SEQ ID NO: 11 (encoding amino acid numbers 1 to 395 in SEQ ID NO: 12), the nucleotide sequence of the nucleotide numbers 544 to 1710 in SEQ ID NO: 11 (encoding amino acid numbers 7 to 395 in SEQ ID NO: 12), or the nucleotide sequence of the nucleotide numbers 568 to 1710 in SEQ ID NO: 11 (encoding the amino acid numbers 15 to 395 in SEQ ID NO: 12) is an open reading frame and can be used as the AKO gene.

SEQ ID NO: 13 is the nucleotide sequence of the region including the thrA gene of Vibrio natriegens. It is estimated that, in SEQ ID NO: 13, ATG (nucleotide numbers 486 to 488), GTG (nucleotide numbers 591 to 593), or GTG (nucleotide numbers 633 to 635) can be the initiation codon, and TAA (nucleotide numbers 2943 to 2945) can be the stop codon. Therefore, a DNA having the nucleotide sequence of the nucleotide numbers 486 to 2942 in SEQ ID NO: 13 (encoding amino acid numbers 1 to 819 in SEQ ID NO: 14), the nucleotide sequence of the nucleotide numbers 591 to 2942 in SEQ ID NO: 13 (encoding the amino acid numbers 35 to 819 in SEQ ID NO: 14), or the nucleotide sequence of the nucleotide numbers 633 to 2942 in SEQ ID NO: 13 (encoding the amino acid numbers 50 to 819 in SEQ ID NO: 14) is an open reading frame and can be used as the thrA gene.

SEQ ID NO: 15 is the nucleotide sequence of the region including the metL gene of Vibrio natriegens. It is estimated that, in SEQ ID NO: 15, ATG (nucleotide numbers 376 to 378), GTG (nucleotide numbers 487 to 489), or GTG (nucleotide numbers 490 to 492) can be the initiation codon, and TAA (nucleotide numbers 2782 to 2784) can be the stop codon. Therefore, a DNA having the nucleotide sequence of the nucleotide numbers 376 to 2781 in SEQ ID NO: 15 (encoding amino acid numbers 1 to 802 in SEQ ID NO: 16), the nucleotide sequence of the nucleotide numbers 487 to 2781 in SEQ ID NO: 15 (encoding amino acid numbers 38 to 802 in SEQ ID NO: 16), or the nucleotide sequence of the nucleotide numbers 490 to 2781 in SEQ ID NO: 15 (encoding amino acid numbers 39 to 802 in SEQ ID NO: 16) is an open reading frame and can be used as the metL gene.

SEQ ID NO: 17 is the nucleotide sequence of the region including the lysC gene of Vibrio natriegens. It is estimated that, in SEQ ID NO: 17, GTG (nucleotide numbers 1060 to 1062), or ATG (nucleotide numbers 1117 to 1119) can be the initiation codon, and TAA (nucleotide numbers 2410 to 2412) constitutes the stop codon. Therefore, a DNA having the nucleotide sequence of the nucleotide numbers 1060 to 2409 in SEQ ID NO: 17 (encoding amino acid numbers 1 to 450 in SEQ ID NO: 18), or the nucleotide sequence of the nucleotide numbers 1117 to 2409 in SEQ ID NO: 17 (encoding amino acid numbers 20 to 450 in SEQ ID NO: 18) is an open reading frame and can be used as the lysC gene.

SEQ ID NO: 19 is the nucleotide sequence of the region including a putative-AK gene of Vibrio natriegens. It is estimated that, in SEQ ID NO: 19, ATG (nucleotide numbers 344 to 346), ATG (nucleotide numbers 380 to 382), or ATG (nucleotide numbers 470 to 472) can be the initiation codon, and TAA (nucleotide numbers 1766 to 1768) can be the stop codon. Therefore, a DNA having the nucleotide sequence of the nucleotide numbers 344 to 1765 in SEQ ID NO: 19 (encoding amino acid numbers 1 to 474 of SEQ ID NO: 20), the nucleotide sequence of the nucleotide numbers 380 to 1765 in SEQ ID NO: 19 (encoding amino acid numbers 13 to 474 in SEQ ID NO: 20), or the nucleotide sequence of the nucleotide numbers 470 to 1765 of SEQ ID NO: 19 (encoding amino acid numbers 43 to 474 in SEQ ID NO: 20) is an open reading frame and can be used as the putative-AK gene.

The aforementioned AKO gene, thrA gene, metL gene, lysC gene, and putative-AK gene can be a hybridized with a complementary strand of each sequence or a probe prepared from these sequences under stringent conditions and coding for a protein having the aspartokinase activity.

The “stringent conditions” can be conditions where a so-called specific hybrid is formed, and a non-specific hybrid is not formed. Although it is difficult to definitely define these conditions with numerals, examples of the stringent conditions include those where highly homologous DNAs hybridize each other, for example, DNAs not less than 80% homologous, not less than 90% homologous, not less than 95% homologous, not less than 97% homologous, or not less than 99% homologous, hybridize each other, and DNAs less homologous than the above do not hybridize each other, or conditions of washing once, or 2 or 3 times, at salt concentrations and temperature corresponding to washing in typical Southern hybridization, i.e., 1× SSC, 0.1% SDS at 60° C., 0.1× SSC, 0.1% SDS at 60° C., or 0.1× SSC, 0.1% SDS at 68° C. Although length of the probe is appropriately chosen according to the conditions of the hybridization, it is usually 100 by to 1 kbp.

In this specification, the term “homology” can mean “identity”.

The aspartokinase activity can be measured by the method described in Miyajima, R. et al., The Journal of Biochemistry, 63 (2), 139-148 (1968).

The aforementioned AKO gene, thrA gene, metL gene, lysC gene and putative-AK gene are not limited to wild-type genes and can be mutant or artificially modified genes coding for a conservative variant protein having the amino acid sequence encoded by the open reading frame of each gene, but including substitutions, deletions, insertions, additions or the like of one or several amino acids at one or a plurality of positions, so long as the genes code for a protein having the aspartokinase activity.

Although the number of the “one or several” amino acid residues can differ depending on the position in the three-dimensional structure or the types of amino acid residues of the protein, specifically, for example, it can be 1 to 20, 1 to 10, or 1 to 5. The aforementioned substitutions, deletions, insertions, or additions of one or several amino acid residues is a conservative mutation that preserves the normal function of the protein. The conservative mutation can be a mutation wherein substitution takes place mutually among Phe, Trp, and Tyr, if the substitution site is an aromatic amino acid; among Leu, Ile and Val, if it is a hydrophobic amino acid; between Gln and Asn, if it is a polar amino acid; among Lys, Arg and His, if it is a basic amino acid; between Asp and Glu, if it is an acidic amino acid; and between Ser and Thr, if it is an amino acid having a hydroxyl group. Typical examples of a conservative mutation are conservative substitutions, and substitutions considered conservative substitutions include, specifically, substitution of Ser or Thr for Ala, substitution of Gln, His or Lys for Arg, substitution of Glu, Gln, Lys, His or Asp for Asn, substitution of Asn, Glu or Gln for Asp, substitution of Ser or Ala for Cys, substitution of Asn, Glu, Lys, His, Asp or Arg for Gln, substitution of Gly, Asn, Gln, Lys or Asp for Glu, substitution of Pro for Gly, substitution of Asn, Lys, Gln, Arg or Tyr for His, substitution of Leu, Met, Val or Phe for Ile, substitution of Ile, Met, Val or Phe for Leu, substitution of Asn, Glu, Gln, His or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of His, Phe or Trp for Tyr, and substitution of Met, Ile or Leu for Val. The aforementioned amino acid substitutions, deletions, insertions, additions, inversions or the like can be a result of a naturally-occurring mutation or a variation due to an individual difference or difference of species of a microorganism which harbors the AKO, thrA, metL, lysC and putative-AK genes. Such a mutant or modified gene as described above can be obtained by modifying the nucleotide sequence of each open reading frame described above by, for example, site-specific mutagenesis so that the encoded protein includes substitution, deletion, insertion or addition of amino acid residues at a specific position.

As the AKO gene, thrA gene, metL gene, lysC gene and putative-AK gene, genes can be used that code for an amino acid sequence not less than 80% homologous, not less than 90% homologous, not less than 95% homologous, not less than 97% homologous, or not less than 99% homologous, to the entire amino acid sequence encoded by the aforementioned open reading frame of each gene, and code for a protein having the aspartokinase activity. The homology of amino acid sequences and nucleotide sequences may be determined by using, for example, the algorithm BLAST (Proc. Natl. Acad. Sci. USA, 90, 5873 (1993)) created by Karlin and Altschul or FASTA (Methods Enzymol., 183, 63 (1990)). On the basis of the algorithm BLAST, the programs called BLASTN and BLASTX have been developed (refer to www.ncbi.nlm.nih.gov).

The above descriptions concerning conservative variants and genes coding for them, as well as conservative mutation are also applied to enzymes other than aspartokinase and genes coding for them, as well as the FucO protein and the fucO gene described herein.

Plasmids for gene cloning can be those replicable in bacteria such as Escherichia bacteria, and specific examples thereof, and include pBR322, pTWV228, pMW119, pUC 19, and so forth.

As the vector that functions in Vibrio bacteria, any vector autonomously replicable in Vibrio bacteria can be used. As the vector plasmid, any of vector plasmids having ori derived from pUC plasmid, pACYC184 plasmid, or IncQ plasmid can be used. As the marker gene used for the selection, the kanamycin resistance gene derived from Tn903, the chloramphenicol resistance gene derived from Tn9, a streptomycin resistant gene, a tetracycline resistant gene and so forth can be used.

In order to prepare a recombinant DNA by ligating dapA and lysC to a vector that functions in Vibrio bacteria, the vector is digested with restriction enzymes that correspond to the terminuses of a DNA fragment containing dapA and lysC. Ligation is usually performed by using a ligase such as T4 DNA ligase. dapA and lysC can be carried by separate vectors or a single vector.

Examples of DNA coding for a mutant dihydrodipicolinate synthetase which is not subject to feedback inhibition by L-lysine include a DNA coding for such a protein having an amino acid sequence in which the histidine residue at position 118 is replaced by a tyrosine residue. Examples of DNA coding for a mutant aspartokinase which is not subject to feedback inhibition by L-lysine include a DNA coding for an AKIII having an amino acid sequence in which the threonine residue at position 352, the glycine residue at position 323, and the methionine residue at position 318 are replaced by isoleucine, asparagine and isoleucine residues, respectively (for these mutants, see U.S. Pat. Nos. 5,661,012 and 6,040,160). Such mutant DNAs can be obtained by site-specific mutagenesis using PCR or the like.

Wide host-range plasmids RSFD80, pCAB1, and pCABD2 are plasmids containing a mutant dapA gene coding for a mutant dihydrodipicolinate synthase and a mutant lysC gene coding for a mutant aspartokinase (U.S. Pat. No. 6,040,160). Escherichia coli JM109 strain transformed with the plasmid was named AJ12396 (U.S. Pat. No. 6,040,160), and the strain was deposited at the National Institute of Bioscience and Human-Technology, Agency of Industrial Science and Technology, Ministry of International Trade and Industry (currently National Institute of Advanced Industrial Science and Technology, International Patent Organism Depositary) on Oct. 28, 1993 and assigned an accession number of FERM P-13936. The deposit was then converted to an international deposit under the provisions of Budapest Treaty on Nov. 1, 1994 and assigned an accession number of FERM BP-4859. RSFD80 can be obtained from the AJ12396 strain by a conventional method.

The recombinant DNA prepared as described above may be introduced into a Vibrio bacterium by any method that affords sufficient transformation efficiency, and examples thereof include electroporation (Canadian Journal of Microbiology, 43, 197 (1997)).

The DDPS activity and/or the AK activity can also be enhanced by introducing multiple copies of the dapA and/or lysC into a chromosomal DNA of a Vibrio bacterium. Introduction of multiple copies of the dapA and/or lysC into a chromosomal DNA of a Vibrio bacterium can be attained by homologous recombination which targets a sequence present on the chromosomal DNA in multiple copies. Such a sequence present on a chromosomal DNA in multiple copies may be a repetitive DNA or an inverted repeat present on the end of a transposable element. Alternatively, as disclosed in Japanese Patent Laid-open No. 2-109985, multiple copies of dapA and/or lysC can be introduced into the chromosomal DNA by inserting the genes into a transposon and transferring it so that multiple copies of the genes are introduced into the chromosomal DNA. These methods increase the copy numbers of dapA and/or lysC, which leads to enhancement in the DDPS activity and/or the AK activity.

Besides the above-described gene amplification, the enhancement of the DDPS activity and/or the AK activity can also be attained by replacing an expression regulatory sequence such as a promoter of dapA and/or lysC with a potent sequence (Japanese Patent Laid-open No. 1-215280). Examples of potent promoters include the lac promoter, trp promoter, trc promoter, tac promoter, PR promoter, and PL promoter of lambda phage, hybrid promoters of these, and so forth. Replacement with these promoters enhances the expression of dapA and/or lysC, and the DDPS activity and/or the AK activity are thereby increased. Replacement of an expression regulatory sequence may be combined with increase of the copy number of dapA and/or lysC (Science, 1997 Sep. 5, 277 (5331):1453-74; Nucleic Acids Res., 1993 Apr. 11, 21(7):1507-16; Proc. Natl. Acad. Sci. USA, 1983 Jan., 80(1):21-5, International Patent Publications WO98/04715, WO98/17806, U.S. Pat. Nos. 5,830,690, and 5,861,273).

Digestion, ligation, etc. of DNA, preparation of chromosomal DNA, PCR, preparation of plasmid DNA, transformation, design of oligonucleotides to be used as primers and so forth can be performed according to conventional methods well known to those skilled in the art. Such methods are described in Sambrook, J., Fritsch, E. F., and Maniatis, T., “Molecular Cloning A Laboratory Manual, Second Edition”, Cold Spring Harbor Laboratory Press, (1989) and so forth.

In addition to enhancement of the DDPS activity and/or the AK activity, activities of other enzymes involved in L-lysine biosynthesis may be enhanced. Examples of such enzymes include enzymes in the diaminopimelic acid pathway, such as dihydrodipicolinate reductase (dapB), diaminopimelate decarboxylase (lysA), diaminopimelate dehydrogenase (ddh) (International Patent Publication WO96/40934 for those mentioned above), phosphoenolpyruvate carboxylase (ppc, Japanese Patent Laid-open No. 60-87788), aspartate aminotransferase (aspC, Japanese Patent Publication No. 6-102028), diaminopimelate epimerase (dapF, Japanese Patent Laid-open No. 2003-135066), and aspartate semialdehyde dehydrogenase (asd, International Patent Publication WO00/61723); and genes coding for enzymes in the aminoadipic acid pathway such as homoaconitate hydratase (Japanese Patent Laid-open No. 2000-157276). Those mentioned in the parentheses after the enzyme names are gene names (the same shall apply to the following descriptions).

The plasmid pCABD2 contains a mutant dapA gene derived from Escherichia coli and coding for DDPS having a mutation for desensitization to the feedback inhibition by L-lysine, a mutant lysC gene derived from Escherichia coli and coding for AKIII having a mutation for desensitization to the feedback inhibition by L-lysine, the dapB gene derived from Escherichia coli and coding for dihydrodipicolinate reductase, and the ddh gene derived from Brevibacterium lactofermentum and coding for diaminopimelate dehydrogenase (International Patent Publications WO95/16042 and WO01/53459).

The Vibrio bacterium may be a bacterium in which the ability to produce L-lysine is increased by enhancing L-lysine secretion activity. For example, by increasing the expression of the ybjE gene, or increasing the expression of the lysE gene, the L-lysine secretion activity can be enhanced (Japanese Patent Laid-open No. 2005-237379, International Patent Publication WO97/23697).

Furthermore, in the bacterium of the presently disclosed subject matter, the activity of an enzyme that catalyzes a reaction which branches off from the L-lysine biosynthesis pathway to produce a compound other than L-lysine, or the activity of an enzyme that negatively effects L-lysine synthesis or accumulation, can be decreased or deleted. Examples of such an enzyme for the L-lysine production include homoserine dehydrogenase, malic enzyme, and so forth, and strains in which activities of such enzymes are decreased or deficient can be constructed by referring to International Patent Publications WO95/23864, WO96/17930, and WO2005/010175.

Activities of these enzymes can be decreased or deleted by introducing a mutation into the genes encoding the enzymes on the genome that results in the activities of the enzymes being decreased or deleted. A conventional mutagenesis or genetic engineering techniques can be used for this purpose. Such introduction of a mutation can be attained by, for example, eliminating the genes coding for the enzymes on the genome by gene recombination or by modifying an expression regulatory sequence such as a promoter or Shine-Dalgamo (SD) sequence. In addition, it can also be attained by introducing a mutation which results in an amino acid substitution (missense mutation) into the region coding for the enzymes on the genome, introducing a stop codon (nonsense mutation), introducing a frame shift mutation that adds or deletes one or two nucleotides, or deleting a part or whole region of the gene (Journal of Biological Chemistry, 272, 8611-8617 (1997); Proc. Natl. Acad. Sci. USA, 95, 5511-5515 (1998), J. Biol. Chem., 266, 20833-20839 (1991)). Furthermore, the activities of the enzymes can also be decreased or eliminated by constructing a mutant gene coding for a mutant enzyme in which all or a part of the coding region is deleted, and then replacing the normal gene on the genome with the mutant gene by homologous recombination or the like, or introducing a transposon or an IS factor into the gene.

For example, such a method as described below is used to introduce a mutation that decreases or eliminates the activities of the above-mentioned enzymes by genetic recombination. A mutant gene is prepared by modifying a partial sequence of a target gene so that it does not produce an enzyme that can function normally. Then, a Vibrio bacterium is transformed with a DNA containing the mutant gene to cause recombination of a gene on the genome with the mutant gene, and thereby the target gene on the genome is replaced with the mutant gene. Examples of this type of gene substitution using homologous recombination include the method using a linear DNA such as the method called “Red-driven integration” (Datsenko, K. A, and Wanner, B. L., Proc. Natl. Acad. Sci. USA, 97:6640-6645 (2000), and a method which is a combination of the Red-driven integration and an excision system derived from λ phage (Cho, E. H., Gumport, R. I., Gardner, J. F., J. Bacteriol., 184, 5200-5203 (2002), refer to International Patent Publication WO2003/010175), a method using a plasmid containing a temperature-sensitive replication origin (U.S. Pat. No. 6,303,383; Japanese Patent Laid-open No. 05-007491), and so forth. Such site-specific mutation as mentioned above based on gene substitution utilizing homologous recombination can also be performed by using a plasmid that is not able to replicate in a host cell.

The aforementioned method for reducing enzyme activity can also be applied to decrease the activity of the FucO protein as described below.

<3> Decrease of Activity of Protein Encoded by fucO Gene

The bacterium of the presently disclosed subject matter can be obtained by modifying such a bacterium belonging to the genus Vibrio and having an ability to produce L-lysine as described above so that activity of the protein encoded by the fucO gene (hereafter the protein is also described as “FucO”) is reduced. In breeding of the Vibrio bacterium, either imparting the ability to produce L-lysine or reducing the activity of the FucO protein may be performed first. Although the bacterium may be modified so that the activity of the protein encoded by the fucO gene is reduced as compared to a wild-type strain or a non-modified strain, it is more desirable that it further has improved ability to accumulate L-lysine as compared to a wild-type strain or a non-modified strain.

In the presently disclosed subject matter, the fucO gene means a homologue of the fucO gene of E. coli obtained from a Vibrio bacterium, and examples include, for example, a gene having the nucleotide sequence of SEQ ID NO: 1. The nucleotide sequence of SEQ ID NO: 1 is the nucleotide sequence of the fucO gene of the V. natriegens 28-15 strain. The amino acid sequence encoded by this gene is shown in SEQ ID NO: 2.

The fucO gene can be homologous to the fucO gene of E. coli. The fucO gene of the E. coli MG1655 strain is registered at GenBank and codes for L-1,2-propanediol oxidoreductase (complementary strand of GenBank NC_{—}000913.2, nucleotide numbers 2929887 to 2931038).

Moreover, the fucO gene includes the eutG gene of Vibrio parahaemolyticus and a gene which is homologous to that gene. The eutG gene product of the V. parahaemolyticus RIMD 2210633 strain is registered at the GenPept database (www.ncbi.nlm.nih.gov/protein/NP_{—}797614.1?report=genpept) as an iron-containing alcohol dehydrogenase (NP_{—}797614). Moreover, the eutG gene is registered at GenBank as a gene encoding an iron-containing alcohol dehydrogenase (NC_{—}004603.1, nucleotide numbers 1301840 to 1303159).

The homology between the fucO gene product of E. coli and the eutG gene product of E. coli in terms of amino acid composition is 77.3% (analyzed by using Genetix ver. 9.0.3 produced by Molecular Devices, Inc.).

A homologue of the fucO gene of a Vibrio bacterium can be obtained by, for example, searching for a gene having high homology to a gene having the nucleotide sequence of SEQ ID NO: 1, the fucO gene of E. coli, or the eutG gene, using BLAST (blast.genome.jp/). The degree of homology is not particularly limited so long as accumulation of L-lysine is improved by reduction of the activity of the expression product of the gene, but the homology can be, for example, 80% or more, 90% or more, 95% or more, 97% or more, or 99% or more, with respect to the entire amino acid sequence.

Examples of proteins encoded by the fucO gene include proteins having the amino acid sequence of SEQ ID NO: 2. However, the protein may be a conservative variant so long as the function of the protein is not changed.

Furthermore, the fucO gene may be a DNA which can be hybridized with the nucleotide sequence of SEQ ID NO: 1, or a probe that can be prepared from that nucleotide sequence under stringent conditions, so long as it codes for the FucO protein. The “stringent conditions” have the same meaning as that described above.

The expression “activity of the protein encoded by the fucO gene is reduced” can mean that the function of the FucO protein encoded by the fucO gene is attenuated or deleted due to introduction of a mutation into the coding region of the fucO gene attained by using a drug or genetic engineering, and also can mean that expression or translation of the fucO gene is reduced due to introduction of a mutation into an expression control region of the fucO gene or the like, and thus the amount of the FucO protein is reduced. Furthermore, the activity of the FucO protein can be reduced by the so-called gene disruption, i.e., deletion of a part of the fucO gene or the entire fucO gene on a chromosome. Reduction of the activity includes complete elimination of the activity.

In order to reduce the activity of the FucO protein, specifically, the methods for reducing an enzyme activity described for impartation of the ability to produce L-lysine can be used.

<4> Method for producing L-lysine

The method of the presently disclosed subject matter is a method of culturing the bacterium of the presently disclosed subject matter in a medium to produce and accumulate L-lysine in the medium or cells of the bacterium, and collecting L-lysine from the medium or the cells.

As the medium, media conventionally used for the production of L-amino acids by fermentation using microorganisms can be used. That is, usual media containing a carbon source, a nitrogen source, inorganic ions, and optionally other organic components as required can be used. As the carbon source, saccharides such as glucose, sucrose, lactose, galactose, fructose, and hydrolysates of starches; alcohols such as glycerol and sorbitol; and organic acids such as fumaric acid, citric acid and succinic acid can be used. In a particular example, glycerol can be used as the carbon source.

Glycerol may be used at any concentration so long as a concentration suitable for production of L-lysine is chosen. When glycerol is used as a sole carbon source in the medium, it can be contained in the medium in an amount of about 0.1 to 50 w/v %, about 0.5 to 40 w/v %, or about 1 to 30% w/v %. Glycerol can also be used in combination with other carbon sources such as glucose, fructose, sucrose, blackstrap molasses and starch hydrolysate. In this case, although glycerol and other carbon sources may be mixed at an arbitrary ratio, the ratio of glycerol in the carbon source can be 10% by weight or more, 50% by weight or more, or 70% by weight or more. Other carbon sources can be saccharides such as glucose, fructose, sucrose, lactose, galactose, blackstrap molasses, starch hydrolysate and a sugar solution obtained by hydrolysis of biomass, alcohols such as ethanol, and organic acids such as fumaric acid, citric acid and succinic acid. Among these, glucose is one example.

Although the initial concentration of glycerol at the time of starting the culture can be as described above, glycerol can be supplemented with consumption of glycerol during the culture.

Glycerol can be pure glycerol or crude glycerol. Crude glycerol can refer to industrially produced glycerol which can contain impurities. Crude glycerol is industrially produced by contacting fats and oils with water under a high temperature and high pressure to hydrolyze them, or by the esterification reaction for biodiesel fuel production. Biodiesel fuel refers to aliphatic acid methyl esters produced from fats and oils and methanol by a transesterification reaction, and crude glycerol is produced as a by-product of this reaction (refer to Fukuda, H., Kondo, A., and Noda, H., 2001, J. Biosci. Bioeng., 92, 405-416). In the biodiesel fuel production process, the alkaline catalyst method is used for the transesterification in many cases, and acids are added for neutralization. Therefore, crude glycerol of a purity of about 70 to 95% by weight, containing water and impurities, is produced. Crude glycerol produced in the biodiesel fuel production process contains, in addition to water, residual methanol, salts of alkali such as NaOH as a catalyst, and an acid used for neutralizing the alkali, such as K₂SO₄, as impurities. Although it depends on manufacturers and production methods, the amount of these salts and methanol can reach several percent. The crude glycerol can contain ions derived from the alkali and the acid used for neutralization thereof, such as sodium ions, potassium ions, chloride ions, and sulfate ions, in an amount of 2 to 7%, 3 to 6%, or 4 to 5.8%, based on the weight of the crude glycerol. Although methanol may not be present as an impurity, it can be present in an amount of 0.01% or less.

The crude glycerol may further contain trace amounts of metals, organic acids, phosphorus, aliphatic acids, and so forth. Examples of the organic acids include formic acid, acetic acid, and so forth, and although they may not be present as impurities, they can be present in an amount of 0.01% or less. Examples of the trace metals contained in the crude glycerol include trace metals required for growth of the microorganism, such as magnesium, iron, calcium, manganese, copper, zinc, and so forth. Magnesium, iron, and calcium can be present in an amount of 0.00001 to 0.1%, 0.0005 to 0.1%, 0.004 to 0.05%, or 0.007 to 0.01%, in terms of the total amount based on the weight of the crude glycerol. Manganese, copper, and zinc can be present in an amount of 0.000005 to 0.01%, 0.000007 to 0.005%, or 0.00001 to 0.001%, in terms of the total amount.

The purity of the crude glycerol may be 10% or higher, 50% or higher, 70% or higher, or 80% or higher. As long as the impurities are within the aforementioned range, the purity of the glycerol may be 90% or higher.

When crude glycerol is used, it can be added to the medium depending on the purity of the glycerol so that the amount of glycerol is within the concentration range described above. Both glycerol and the crude glycerol may be added to the medium.

As the nitrogen source, inorganic ammonium salts such as ammonium sulfate, ammonium chloride, and ammonium phosphate, organic nitrogen such as soybean hydrolysate, ammonia gas, aqueous ammonia, and so forth can be used. As organic trace nutrient sources, the medium can contain the required substances such as vitamin B1 and L-homoserine, yeast extract, and so forth in appropriate amounts. Other than the above, potassium phosphate, magnesium sulfate, iron ions, manganese ions and so forth are added in small amounts, as required. In addition, the medium may be either a natural medium or a synthetic medium, so long as the medium contains a carbon source, a nitrogen source, and inorganic ions, and other organic trace ingredients as required.

The culture can be performed for 1 to 7 days under aerobic conditions. The culture temperature can be 24 to 37° C., and the pH during the culture can be 5 to 9. For pH adjustment, inorganic or organic acidic or alkaline substances, ammonia gas, and so forth can be used.

When a basic amino acid such as L-lysine is produced, the production can be performed by controlling the pH of the medium during fermentation to be 6.5 to 9.0, and the pH of the medium at the end of the culture can be 7.2 to 9.0, and then a culture period can be provided when the medium contains 20 mM or more of bicarbonate ions and/or carbonate ions, so that these bicarbonate ions and/or carbonate ions serve as counter ions of the basic amino acid, and the objective basic amino acid can then be collected (Japanese Patent Laid-open No. 2002-65287, U.S. Patent Published Application No. 2002/0025564, EP 1813677 A).

When a microorganism having a basic amino acid-producing ability is cultured in a medium under aerobic conditions, carbonate ions, bicarbonate ions, or both can be used as major counter ions of the basic amino acid. To provide carbonate ions and/or bicarbonate ions in the medium in an amount required to serve as counter ions of the basic amino acid, it is known that the pH of the medium can be controlled to be 6.5 to 9.0, or 6.5 to 8.0 in another example, during the culture, and can be 7.2 to 9.0 at the end of the culture, and the pressure in the fermentation tank can be controlled so that it is positive during fermentation, or carbon dioxide or a mixed gas containing carbon dioxide can be supplied into the medium (Japanese Patent Laid-open No. 2002-65287, U.S. Patent Published Application No. 2002/0025564, EP 1813677 A).

L-lysine can be collected from a fermentation solution by a combination of the following conventionally known methods: use of an ion-exchange resin (Nagai, H. et al., Separation Science and Technology, 39(16), 3691-3710), precipitation, membrane separation (Japanese Patent Laid-open Nos. 9-164323 and 9-173792), crystallization (International Patent Publications WO2008/078448, WO2008/078646), and so forth. When L-lysine is accumulated in cells, the cells can be disrupted with, for example, ultrasonication or the like, and L-lysine can be collected by the ion exchange resin method or the like from supernatant obtained by removing the cells from the cell-disrupted suspension by centrifugation.

The collected L-lysine may contain bacterial cells, medium components, moisture, and by-product metabolites of the bacterium in addition to L-lysine. Purity of the collected L-lysine can be 50% or higher, 85% or higher, or even 95% or higher (Japanese Patent No. 1214636, U.S. Pat. Nos. 5,431,933, 4,956,471, 4,777,051, 4,946,654, 5,840,358, 6,238,714, U.S. Patent Published Application No. 2005/0025878).

Furthermore, when L-lysine precipitates in the medium, it can be collected by centrifugation, filtration or the like. L-Lysine precipitated in the medium and L-lysine dissolved in the medium may be isolated together after the L-lysine dissolved in the medium is crystallized.

### EXAMPLES

Hereinafter, the present invention will be more specifically explained with reference to the following non-limiting examples.

### Example 1

### Construction of fucO Gene-Disrupted V. natriegens

The fucO gene on the chromosome of lysine-producing bacterium, V. natriegens 28-15, was disrupted.

First, the sacB gene of Bacillus subtilis was amplified by PCR using pDNR-Dual Doner vector (Clontech, USA) having the sacB gene of Bacillus subtilis as a template DNA, and synthetic DNAs having the sequences of SEQ ID NOS: 3 and 4 as primers. The sacB gene encodes levan sucrase, and can be used to efficiently select a strain in which the vector portion is eliminated from the chromosome (Schafer, A. et al., Gene, 145, 69-73 (1994)). That is, if levan sucrase is expressed, sucrose is assimilated, and the produced levan lethally acts on bacteria, so that they cannot grow. Therefore, if a strain in which a vector carrying a levan sucrase gene still remains on the chromosome is cultured on a plate containing sucrose, it cannot grow, and thus only a strain in which the vector is eliminated can be selected on the plate containing sucrose.

The amplified sacB gene fragment and pUT399 were digested with the restriction enzyme EcoRI and ligated to obtain a vector pUT_sacB. pUT399 is a plasmid having the replication origin of R6K, and containing the mob region required for conjugative transfer, and it cannot replicate in a strain which does not have the pir gene (U.S. Pat. No. 7,090,998). Then, PCR was performed by using the chromosomal DNA of V. natriegens 28-15 as a template DNA and synthetic DNAs having the sequences of SEQ ID NOS: 5 and 6 or SEQ ID NOS: 7 and 8 as primers, sequences of about 800 by on both sides of the fucO gene were amplified, respectively. Then, PCR was performed again by using a mixture of two kinds of the obtained PCR products as a template DNA, and synthetic DNAs of the sequences of SEQ ID NOS: 5 and 7 as primers. The obtained PCR product and pUT_sacB were digested with the restriction enzymes SalI and SphI, and ligated to obtain a vector pUT_sacB_fucOFR. pUT_sacB_fucOFR is a plasmid for disrupting the fucO gene, lacking a part of the coding region of fucO. And pUT_sacB_fucOFR was introduced into the E. coli S 17-1 (λpir+, available from Biomedal, R. Simon., et al., Bio/Technology, 1:784-791 (1983)) to obtain S17-1/pUT_sacB_fucOFR.

Then, S17-1/pUT_sacB_fucOFR was inoculated into 1.5 ml of the LB medium (10 g/L of Bacto tryptone, 5 g/L of Bacto yeast extract, 5 g/L of NaCl, and 40 mg/L of chloramphenicol, pH 7.0), and cultured at 37° C. for 18 hours. The cells were collected by centrifuging the culture medium (at 5000 rpm for 5 minutes), and suspended in 50 μl of fresh LB medium. This cell suspension was inoculated onto the LB-NaCl agar medium (10 g/L of Bacto tryptone, 5 g/L of Bacto yeast extract, 30 g/L of NaCl, 0.4 g/L of MgSO₄, and 20 g/L of agar, pH 7.0) in the shape of circle having a diameter of 2 cm, and left to dry at room temperature for 30 minutes. In parallel, V. natriegens 28-15 was inoculated into 1.5 ml of the LB-NaCl medium (10 g/L of Bacto tryptone, 5 g/L of Bacto yeast extract, 30 g/L of NaCl, and 0.4 g/L of MgSO₄, pH 7.0), and cultured at 37° C. for 18 hours. The cells were collected by centrifuging the culture medium (at 6000 rpm for 2 minutes), and suspended in 50 μl of fresh LB-NaCl medium. This cell suspension was inoculated on the site at which S17-1/pUT_sacB_fucOFR had been previously inoculated in the shape of circle, and left to dry at room temperature for 30 minutes. Then, the cells were left at 37° C. for 4 hours so that pUT_sacB_fucOFR harbored by S17-1/pUT_sacB_fucOFR would transfer to V. natriegens 28-15 by conjugative transfer.

The obtained bacterial cells were scraped together, and suspended in 1 ml of the LB-NaCl medium, then the total volume of the suspension was inoculated on a TCBS agar medium (5 g/L of yeast extract, 10 g/L of peptone, 17 g/L of sucrose, 10 g/L of sodium thiosulfate (pentahydrate), 10 g/L of sodium citrate (dihydrate), 3 g/L of sodium cholate, 1 g/L of ferric citrate, 10 g/L of sodium chloride, 5 g/L of bovine bile powder, 0.04 g/L of bromothymol blue, 0.05 g/L of thymol blue, 15 g/L of agar, and 10 mg/L of chloramphenicol, NISSUI PHARMACEUTICAL CO., LTD., Japan), and culture was performed at 37° C. for 18 hours to allow for formation of yellow colonies. On the TCBS medium, E. coli cannot proliferate. Furthermore, pUT_sacB_fucOFR cannot replicate in V. natriegens 28-15. Therefore, the obtained colonies were a V. natriegens 28-15 strain that had acquired chloramphenicol resistance by introduction of pUT_sacB_fucOFR into the chromosome.

That strain was inoculated in the LB-NaCl medium supplemented with 10% (w/v) sucrose, and colonies of V. natriegens 28-15 in which the fucO gene on the chromosome was disrupted along with elimination of pUT_sacB_fucOFR from the chromosome (V. natriegens 28-15 ΔfucO) were obtained. The disruption of the fucO gene was confirmed by PCR using chromosomal DNA extracted from the obtained strain in a conventional manner as a template DNA, and DNAs having the sequences of SEQ ID NOS: 9 and 10 as primers.

### Example 2

L-Lysine decomposition activity of V. natriegens 28-15 ΔfucO was examined.

pCABD2 (U.S. Pat. No. 6,040,160) was introduced into V. natriegens 28-15 ΔfucO and V. natriegens 28-15 by electroporation. Electroporation was performed by using Gene Pluser Xcell (BioRad, USA) with pulse conditions of 9 kV/cm, 25 μF, and 200 Ω.

The obtained V. natriegens 28-15 ΔfucO/pCABD2 and V. natriegens 28-15/pCABD2 were each cultured on the LB-NaCl agar medium (containing 500 mg/L of streptomycin) at 37° C. for 10 hours. The obtained cells were scraped together, inoculated into 20 ml of the MS medium (containing 500 mg/L of streptomycin) in a Sakaguchi flask (volume: 500 ml) at OD660 of 0.1, and cultured at 37° C. with shaking. Each strain was cultured in three different flasks. The medium was periodically sampled in a volume of 1 ml, and OD660 as well as glucose concentration and L-lysine concentration in the medium were measured. OD660 was measured for the culture medium diluted 51 times with a 2% (w/v) NaCl aqueous solution by using a spectrophotometer DU-800 (Beckman Coulter, USA). The glucose concentration and L-lysine concentration in the medium were measured by using Biotech Analyzer AS-300 (Sakura Seiki Co., Ltd., Japan).

MS culture medium
Final concentration

| | | |
|---|---|---|
| Glucose | 40 | g/L (separately sterilized) |
| MgSO₄•7H₂O | 1 | g/L (separately sterilized) |
| (NH₄)₂SO₄ | 16 | g/L |
| KH₂PO₄ | 1 | g/L |
| Yeast extract | 2 | g/L |
| FeSO₄ | 0.01 | g/L |
| MnSO₄ | 0.01 | g/L |
| CaCO₃ | 30 | g/L (separately sterilized) |
| NaCl | 15 | g/L |

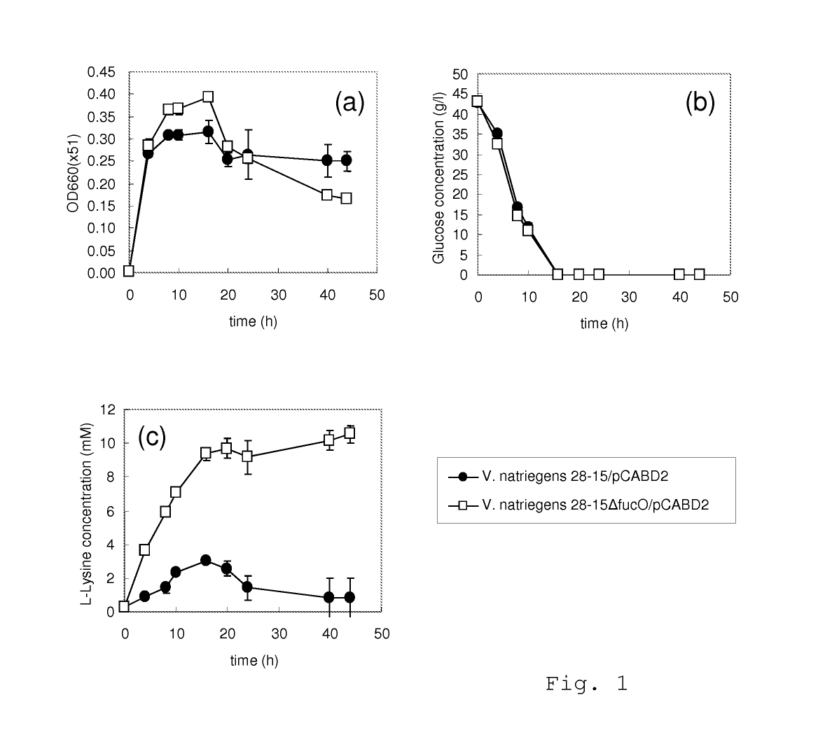

As a result, with V. natriegens 28-15 ΔfucO/pCABD2, accumulation of 10 mM L-lysine was observed, and decomposition of L-lysine was not observed after the glucose was completely consumed (FIG. 1). On the other hand, with the strain in which the fucO gene was not disrupted, although accumulation of 3 mM L-lysine was observed at most, decomposition of L-lysine was observed after that. That is, with the fucO-deficient strain, more than 3 times of L-lysine accumulated, and decomposition of L-lysine was also suppressed, as compared to the strain in which the fucO gene was not deleted.

As described above, it has been suggested that the fucO gene is involved in decomposition of L-lysine in V. natriegens. Therefore, the L-lysine decomposition pathway of V. natriegens may differ from the pathway through which cadaverine is generated from L-lysine.

### Example 3

Then, with V. natriegens 28-15 ΔfucO/pCABD2, L-lysine was produced using a high salt concentration with glucose as the carbon source. V. natriegens 28-15 ΔfucO/pCABD2 was cultured on the LB-NaCl agar medium (containing 500 mg/L of streptomycin) at 37° C. for 8 hours. The obtained cells were scraped together, inoculated into 20 ml of the MS medium (containing 500 mg/L of streptomycin, and 0 to 8% (w/v) NaCl) in a Sakaguchi flask (volume: 500 ml) at OD660 of 0.1, and cultured at 37° C. with shaking. The strain was cultured in three different flasks, each having a different salt concentration. The medium was periodically sampled in a volume of 1 ml, and OD660 and the glucose and L-lysine concentrations in the medium were also periodically measured. OD660 was measured for the culture medium diluted with 51 times with a 2% (w/v) NaCl aqueous solution by using a spectrophotometer DU-800 (Beckman Coulter, USA). The glucose and L-lysine concentrations in the medium were measured by using Biotech Analyzer AS-300 (Sakura Seiki Co., Ltd., Japan).

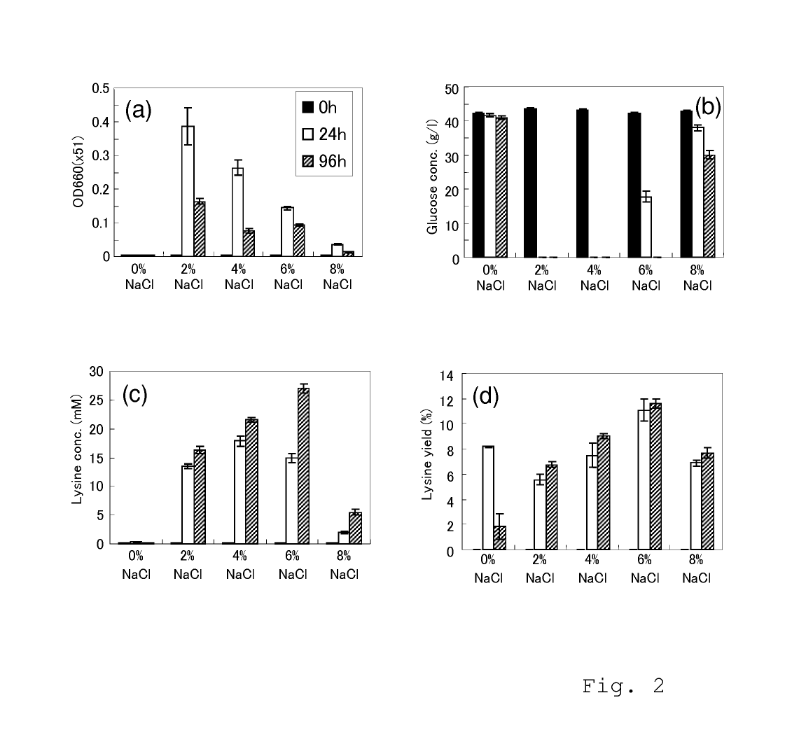

As a result, when 6% (w/v) NaCl was added to the culture, 27 mM L-lysine accumulated at most, and the L-lysine yield based on consumed saccharide at that time was 12% (w/w) (FIG. 2).

### Example 4

Then, with V. natriegens 28-15 ΔfucO/pCABD2, L-lysine was produced by using glycerol as the carbon source. Specifically, V. natriegens 28-15 ΔfucO/pCABD2 was cultured on the LB-NaCl agar medium (containing 500 mg/L of streptomycin) at 37° C. for 8 hours. The obtained cells were scraped together, inoculated into 20 ml of the MS medium (containing 500 mg/L of streptomycin, and 2% or 4% (w/v) NaCl) at OD660 of 0.1, and cultured at 37° C. with shaking. As the glycerol of the carbon source, glycerol produced by Junsei Chemical Co., Ltd., Japan was used. The strain was cultured in three different flasks for each salt concentration. The medium was periodically sampled in a volume of 1 ml, and OD660 and the glycerol and L-lysine concentrations in the medium were also periodically measured. OD660 was measured for the culture medium diluted with 51 times with a 2% (w/v) NaCl aqueous solution by using a spectrophotometer DU-800 (Beckman Coulter, USA). The glycerol concentration in the medium was measured by using Biosensor BF-5 (Oji Scientific Instruments Co., Ltd., Japan), and the L-lysine concentration was measured by using Biotech Analyzer AS-300 (Sakura Seiki Co., Ltd., Japan).

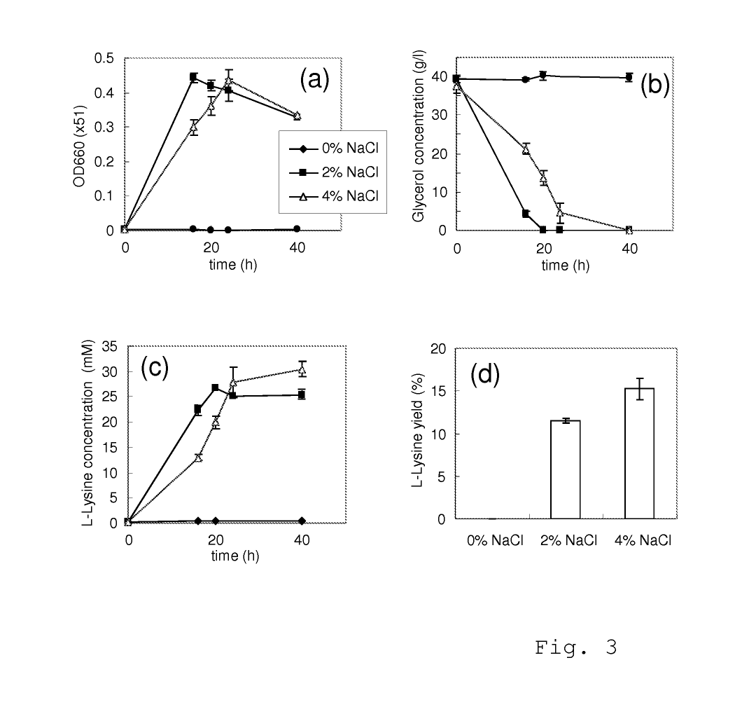

As a result, when 4% (w/v) NaCl was added, 30 mM L-lysine accumulated at most, and the L-lysine yield based on consumed saccharide was 15% (w/w) (FIG. 3).

### Example 5

In the same manner as that of Example 1 used for obtaining V. natriegens 28-15 ΔfucO, except that a V. natriegens wild-type strain, ATCC 14048 strain (NBRC 15636, AJ13670), was used instead of the V. natriegens 28-15 strain, a fucO gene-disrupted strain, VLD01, was obtained.

### Example 6

With V. natriegens 28-15 ΔfucO/pCABD2, VLD01/pCABD2, and V. natriegens ATCC 14048/pCABD2, L-lysine production was performed by using glycerol as the carbon source in the MS4Y medium, which is the MS culture excessively supplemented with yeast extract. First, each strain was cultured on the LB-NaCl agar medium (containing 500 mg/L of streptomycin) at 37° C. for 8 hours. The obtained cells were scraped together, inoculated into 20 ml of the MS4Y medium at OD660 of 0.1, and cultured at 37° C. with shaking. As the glycerol of the carbon source, glycerol produced by Junsei Chemical Co., Ltd., Japan was used. Each strain was cultured in two flasks. The medium was periodically sampled in a volume of 0.5 ml, and OD660 and the glycerol and L-lysine concentrations in the medium were also periodically measured. OD660 was measured for the culture medium diluted with 50 times with 1 N HCl aqueous solution by using a spectrophotometer DU-800 (Beckman Coulter, USA). The glycerol concentration in the medium was measured by using Biosensor BF-5 (Oji Scientific Instruments Co., Ltd., Japan), and the L-lysine concentration was measured by using Biotech Analyzer AS-300 (Sakura Seiki Co., Ltd., Japan).

MS4Y medium
Final concentration

| | | |
|---|---|---|
| Glycerol | 40 | g/L (separately sterilized) |
| MgSO₄•7H₂O | 1 | g/L (separately sterilized) |
| (NH₄)₂SO₄ | 24 | g/L |
| KH₂PO₄ | 1 | g/L |
| Yeast extract | 8 | g/L |
| FeSO₄ | 0.01 | g/L |
| MnSO₄ | 0.01 | g/L |
| CaCO₃ | 30 | g/L (separately sterilized) |
| NaCl | 20 | g/L (separately sterilized) |

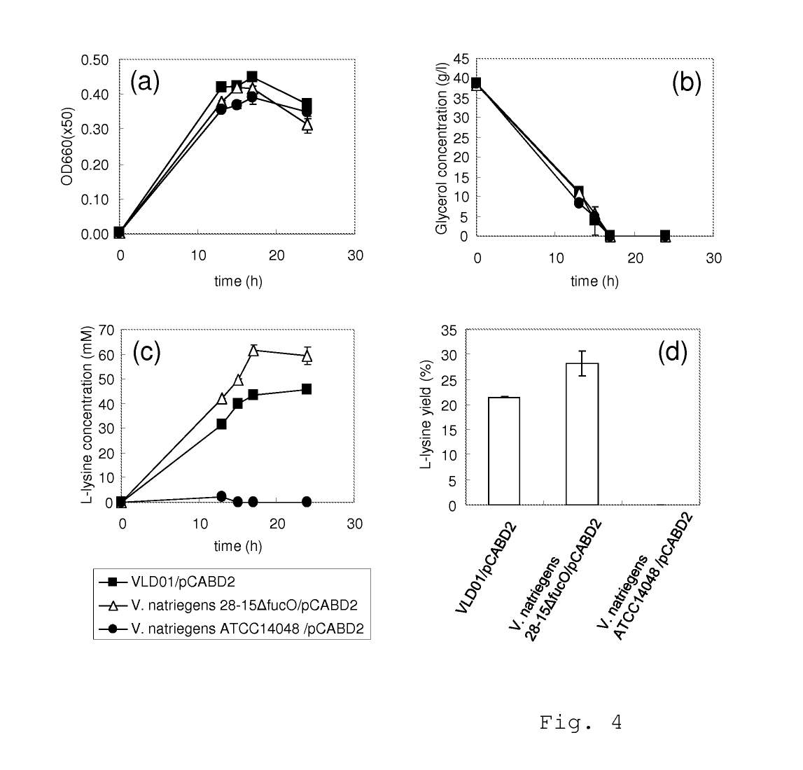

In the medium excessively supplemented with yeast extract, V. natriegens 28-15 ΔfucO/pCABD2 produced 61 mM L-lysine (FIG. 4). In this case, the L-lysine yield based on consumed glycerol was 28% (w/w). Furthermore, accumulation of 45 mM L-lysine was also observed with VLD01/pCABD2, and the L-lysine yield based on consumed glycerol was 21% (w/w). With V. natriegens ATCC 14048/pCABD2, accumulation of L-lysine was not substantially observed.

Explanation of Sequence Listing

SEQ ID NO: 1: Nucleotide sequence of fucO gene of V. natriegens

SEQ ID NO: 2: Amino acid sequence encoded by fucO gene of V. natriegens

SEQ ID NO: 3: Nucleotide sequence of PCR primer for amplifying sacB gene

SEQ ID NO: 4: Nucleotide sequence of PCR primer for amplifying sacB gene

SEQ ID NO: 5: Nucleotide sequence of PCR primer for amplifying 0.8 kbp sequences on both sides of fucO gene

SEQ ID NO: 6: Nucleotide sequence of PCR primer for amplifying 0.8 kbp sequences on both sides of fucO gene

SEQ ID NO: 7: Nucleotide sequence of PCR primer for amplifying 0.8 kbp sequences on both sides of fucO gene

SEQ ID NO: 8: Nucleotide sequence of PCR primer for amplifying 0.8 kbp sequences on both sides of fucO gene

SEQ ID NO: 9: Nucleotide sequence of PCR primer for confirming disruption of fucO gene on chromosome

SEQ ID NO: 10: Nucleotide sequence of PCR primer for confirming disruption of fucO gene on chromosome

SEQ ID NO: 11: Nucleotide sequence of AKO gene of V. natriegens

SEQ ID NO: 12: Amino acid sequence encoded by AKO gene of V. natriegens

SEQ ID NO: 13: Nucleotide sequence of thrA gene of V. natriegens

SEQ ID NO: 14: Amino acid sequence encoded by thrA gene of V. natriegens

SEQ ID NO: 15: Nucleotide sequence of metL gene of V. natriegens

SEQ ID NO: 16: Amino acid sequence encoded by metL gene of V. natriegens

SEQ ID NO: 17: Nucleotide sequence of lysC gene of V. natriegens

SEQ ID NO: 18: Amino acid sequence encoded by lysC gene of V. natriegens

SEQ ID NO: 19: Nucleotide sequence of putative-AK gene of V. natriegens

SEQ ID NO: 20: Amino acid sequence encoded by putative-AK gene of V. natriegens

### INDUSTRIAL APPLICABILITY

According to the method of the present invention, L-lysine can be efficiently produced by using a Vibrio bacterium.

In particular, with a Vibrio bacterium deficient in the fucO gene, decomposition of L-lysine accumulated with time is suppressed, and thus L-lysine can be very efficiently accumulated.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

What is claimed is:

1. A method for producing L-lysine, which comprises:
a) culturing a Vibrio bacterium having an ability to produce L-lysine in a medium to produce and accumulate L-lysine in the medium or cells of the bacterium, and
b) collecting L-lysine from the medium or cells, wherein the Vibrio bacterium has been modified so that an activity of a protein encoded by the fucO gene is reduced.

2. The method according to claim 1, wherein the activity of the protein is reduced by introducing a mutation into:
a) a coding region of the fucO gene,
b) an expression control region of the gene, and
c) combinations thereof.

3. The method according to claim 1, wherein the fucO gene on the bacterium's chromosome is disrupted.

4. The method according to claim 1, wherein the protein is selected from the group consisting of:
(A) a protein comprising the amino acid sequence shown in SEQ ID NO: 2, and
(B) a protein comprising the amino acid sequence shown in SEQ ID NO: 2, but wherein one or several amino acid residues are substituted, deleted, inserted or added,
wherein the reduction of the activity in the bacterium improves the ability to produce L-lysine.

5. The method according to claim 1, wherein the fucO gene is a DNA selected from the group consisting of:
(a) a DNA comprising the nucleotide sequence of SEQ ID NO: 1,
(b) a DNA which hybridizes with the nucleotide sequence of SEQ ID NO: 1 or a probe that can be prepared from the nucleotide sequence under stringent conditions, and codes for a protein, wherein the reduction of the activity in the bacterium improves the ability to produce L-lysine.

6. The method according to claim 1, wherein an activity is enhanced of an enzyme selected from the group consisting of dihydrodipicolinate synthase, aspartokinase, dihydrodipicolinate reductase, diaminopimelate dehydrogenase, and combinations thereof.

7. The method according to claim 1, wherein the Vibrio bacterium is Vibrio natriegens.

8. The method according to claim 1, wherein the medium comprises glycerol as a carbon source.

9. A Vibrio bacterium which has an ability to produce L-lysine, and has been modified so that an activity of a protein encoded by the fucO gene is reduced.

10. The Vibrio bacterium according to claim 9, which is Vibrio natriegens.
